(19) United States
(12) Patent Application Publication
(10) Pub. No.: US 2011/0281933 A1
(43) Pub. Date: Nov. 17, 2011

(54) METHODS AND COMPOSITIONS FOR THE MANAGEMENT OF CARDIOVASCULAR DISEASE WITH OLIGONUCLEOTIDES

(75) Inventors: Angel Baldan, St. Louis, MO (US); Tyler Marquart, St. Louis, MO (US); Ryan Allen, Imperial, MO (US)

(73) Assignee: Saint Louis University, Saint Louis, MO (US)

(21) Appl. No.: 13/107,792

(22) Filed: May 13, 2011

Related U.S. Application Data
(60) Provisional application No. 61/334,565, filed on May 13, 2010.

(51) Int. Cl. A61K 31/7088; A61K 31/711; A61P 1/16; A61P 9/00; A61P 1/18; A61P 15/00; A61P 21/00; A61P 1/00; A61K 31/7105; A61P 25/00

(52) U.S. Cl. 514/44 A

(57) ABSTRACT

Disclosed are compositions and methods for treating cardiovascular disease and reducing the adverse effects induced by the administration of statins. In particular, disclosed is the use of antisense compounds to augment the expression of mirR-33 and associated genetic elements. In particular methods of the treatment of cardiovascular disease and the modulation of miR-33 levels is disclosed as well as treatment of the secondary effects including cholestasis, induced by the administration of statins is disclosed. Also disclosed is the treatment of Benign Recurrent Intrahepatic Cholestasis and reverse cholesterol transport. The disclosed methods and compositions may be practiced separately or co-administered with satins to reduce or treat statin induced secondary effects.

### CROSS REFERENCE TO RELATED APPLICATION

This application claims priority to provisional application 61/334,565, filed May 13, 2010, hereby incorporated by reference in its entirety.

### FIELD OF THE INVENTION

The invention relates to methods and composition for modulating cholesterol in a mammal. The invention includes the use of certain oligonucleotides for inhibiting the expression of specific proteins related to cholesterol efflux and synthesis for the purpose of managing cardiovascular disease and also for the purpose of managing secondary effects in patients due to treatment with statins.

### BACKGROUND

Atherosclerosis is a progressive disorder wherein lipid loaded macrophages initially accumulate in the sub-endothelial space. Subsequently, more advance plaques develop that contain extracellular lipid, a necrotic core and inflammatory cells. Such plaques can eventually rupture leading to the formation of thrombi (Lusis, (2000) Nature; 407:233; Glass, and Witzturn, (2001) Cell; 104:503). Together, these changes in the artery wall can result in heart attacks, strokes, and peripheral artery disease, which collectively accounted for >30% of all deaths in the US during the last decade (Rosamond et al., (2007) Circulation; 115:e69)

The development of atherosclerosis and the risk of a myocardial infarct are accelerated by a number of factors including hypercholesterolemia (Kannel, et al, (1979) Ann Intern Med.; 90:85). One such risk factor is the accumulation of LDL-cholesterol- and decrease of HDL-cholesterol. Patients with a low HDL/LDL ratio are at increased risk for heart disease. There is great interest in the development of pharmaceuticals which will increase the HDL/LDL ratio in patients at risk including those with hypercholesterolemic. Statins represent the most common pharmacologic treatment for patients at risk including hypercholesterolernic patients (Baigent et al., (2005) Lancet; 366:1267). Statins inhibit hepatic HMG-CoA reductase, the rate-limiting enzyme in the cholesterol synthesis pathway (Steinberg, (2006) J Lipid Res.; 47:1339). This decrease in sterol synthesis/levels results in increased nuclear localization of SREBP-2, which then promotes the transcription of the LDL-R, ultimately leading to increased clearance of circulating LDL-cholesterol (Brown, and Goldstein, (1997) Cell; 89:331; Goldstein, and Brown, (2009) Arterioscler Thromb Vasc Biol.; 29:431). The administration of statins is known to be associated with adverse side effects or statin induced secondary effects. The most common are raised liver enzymes and muscle problems including rhabdomyolysis. Other possible adverse effects due to statins include cognitive loss, cholestasis, neuropathy, pancreatic and hepatic dysfunction, and sexual dysfunction. An effective method of treating the statin induced secondary effects would be highly desirable.

While decreasing plasma LDL-cholesterol is an effective method of increasing the HDL/LDL ratio an alternative or complementary method is to increase levels of HDL-cholesterol. Drugs which increase HDL-cholesterol will be useful in modulating the HDL/LDL ratio in subjects when administered alone and/or when administered in combination with statins. Recently, there has been much interest in microRNAs (miRNAs). Expression of particular miRNAs has been found to be tissue, developmental, and even disease specific. Recent studies have shown that miRNAs function as key mediators in multiple normal and disease-related biological processes (Coolen, and Bally-Coif, (2009) Curr Opin Neurobiol.; 19:461; van Rooij et al., (2008) Proc Natl Aced Sci USA; 105:13027; Asirvatham, et al, (2008) Mol Immunol; 45:1995). Consequently, the instant invention fills a long felt need for the management of cardiovascular diseases, including the management of statin-induced secondary effects by using methods and compositions that exploit technology related to miRNA dependent gene silencing.

### SUMMARY OF THE INVENTION

Disclosed are methods and compositions for modulating the HDL/LDL ratio in a patient in need using antisense compounds complementary to miR-33.

Disclosed are methods and compositions for treating a statin induced secondary effect in a patient in need by administering an effective amount of an antisense compound complementary to miR-33.

Disclosed are methods and compositions for treating statin induced secondary effects, including but not limited to raised liver enzymes, rhabdomyolysis, cognitive loss, cholestasis, Benign Recurrent Intrahepatic Cholestasis (BRIC), neuropathy, pancreatic, hepatic dysfunction, and sexual dysfunction by administering an effective amount of an antisense compound complementary to miR-33.

Disclosed are methods and compositions for treating Benign Recurrent Intrahepatic Cholestasis not associated with statins by administering an effective amount of an antisense compound complementary to miR-33.

Disclosed are methods and compositions for improving cardiovascular health by increasing reverse cholesterol transport (RCT) by administering an effective amount of an antisense compound complementary to miR-33.

### REFERENCE TO COLOR FIGURES

The application file contains at least one figure executed in color. Copies of this patent application publication with color photographs will be provided by the Office upon request and payment of the necessary fee.

### DESCRIPTION OF THE FIGURES

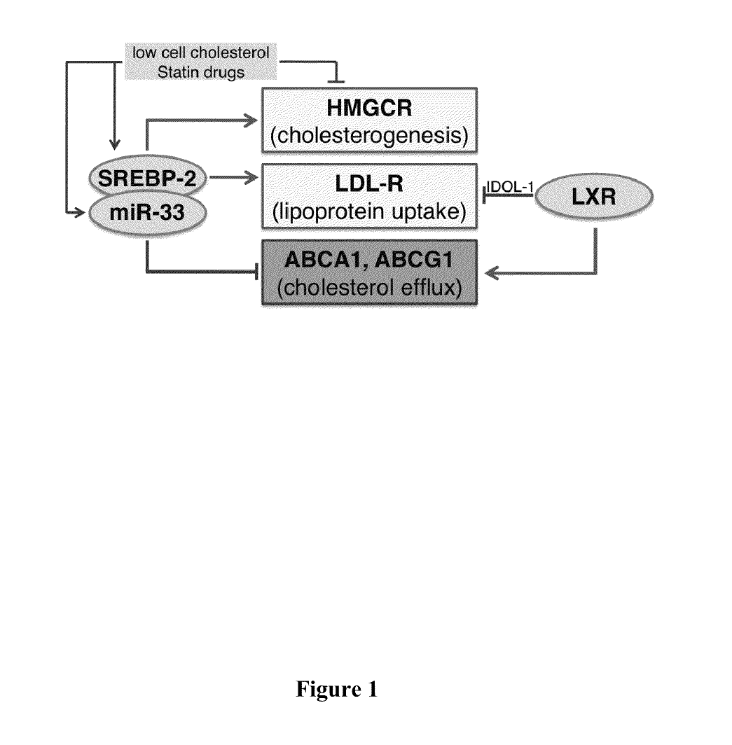

FIG. 1 shows a unified paradigm for cholesterol homeostasis. SREBP-2 and LXR coordinately regulate the positive and negative balances of intracellular cholesterol metabolism. Both pathways are not independent, but intersect through IDOL-1 and miR-33. Statin drugs, or conditions of low intracellular cholesterol, induce both SREBP-2 and the intragenic miR-33 leading to increased synthesis and uptake of sterols, as well as minimizing sterol loss through ABC transporters to exogenous acceptors.

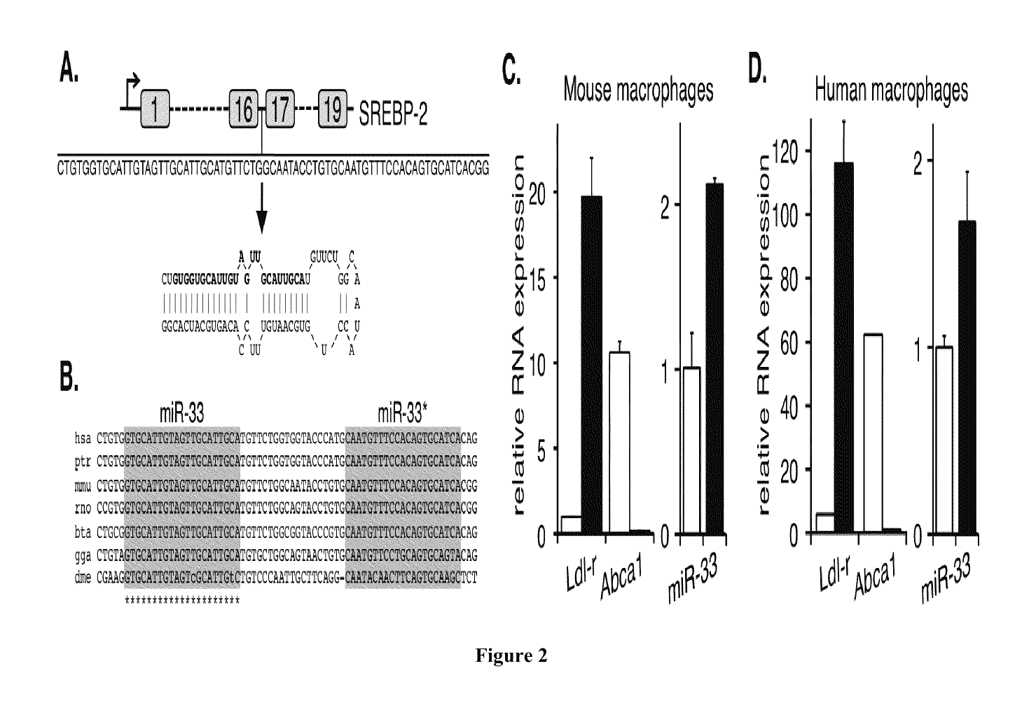

FIG. 2 shows the intragenic miR-33 is encoded within intron 16 of SREBP-2 (A), and both its sequence and genomic position are conserved across evolution (B). Expression of miR-33 and selected SREBP-2 and LXR target genes in human (C) and mouse (D) primary macrophages following 48 h incubation In media containing high (closed bars) or low (open bars) levels of sterols (see Methods). **P<0.01. Data are mean±SD of two independent experiments in triplicate.

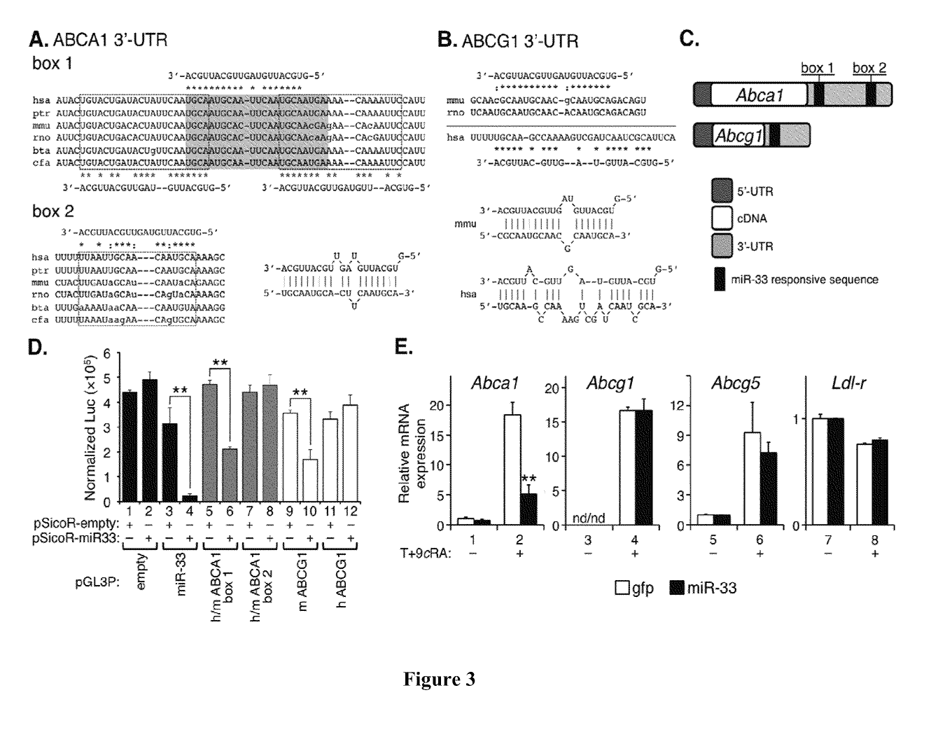

FIG. 3 shows regulation of the LXR targets ABCA1 and ABCG1 by miR-33. (A-C) Evolutionary conserved sequences in the 3′-UTR of ABCA1 and ABCG1 are partially complementary to miR-33. Annealings of miR-33 to some of the sequences are shown. (D) Luciferase activity in HEK293 cells following co-transfection of different constructs containing these putative response elements for miR-33 cloned downstream of the reporter stop codon, co-transfected with or without a miR-33 expression plasmid. Repression of luciferase activity suggests these sequences are physiological targets for miR-33. Deviation from miR-33 complementarity results in loss of regulation by miR-33 (ABCA1 box 2; human ABCG1 sequence). (E) Expression of selected genes in Hep3B human hepatoma cells 48 h after transduction with an empty or a miR-33 adenovirus. Where indicated, cells were incubated for 8 h with LXR:RXR agonists (1 μmol/L T0901317 and 1 μmol/L 9-cis retinoid acid, respectively). **P<0.01. Data are mean±SD of three independent experiments in duplicate.

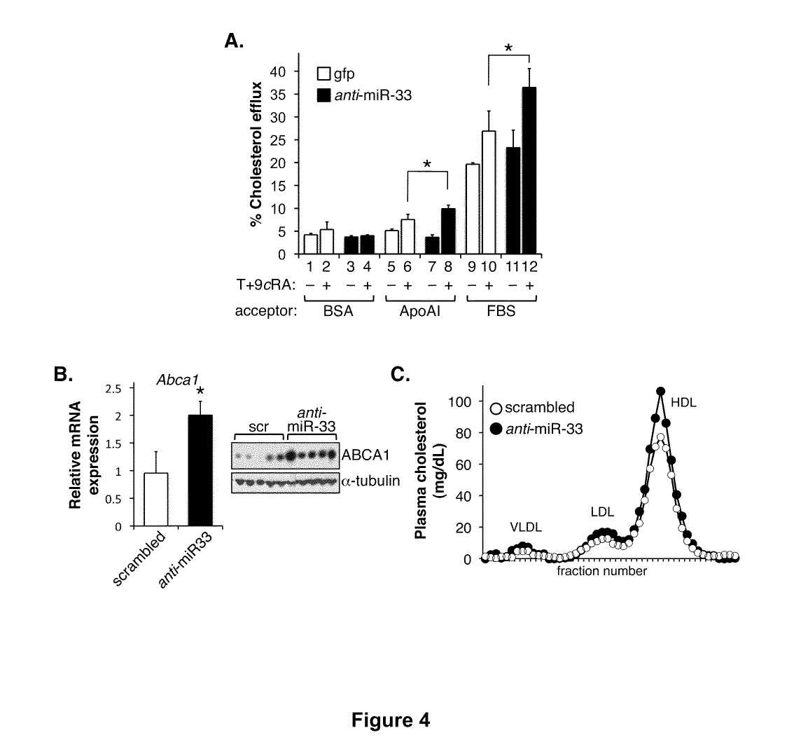

FIG. 4 shows silencing miR-33 increases HDL lipidation. (A) Cholesterol efflux assay in HEK293 cells transfected with scrambling or anti-miR-33 oligonucletide (see Methods for details). After 36 h, the cells were washed and incubated for 16 h in media supplemented with [311]-cholesterol (1 mCi/mL) in the presence or absence of LXR:RXR ligands, as described in FIG. 3. After 16 h, fresh media supplemented with BSA (0.2%). ApoAI (15 mg/mL) or FBS (20%) was added to the cells. Radioactivity in the media and in cell lysates was measured 6 h later. The % efflux is expressed as dpm in the media vs. total dpm (media+cells). *P<0.01. Data are mean±SD of two independent experiments in quadruplicate. Data are mean±SD. (B) Mice (8-10 weeks old male C57Bl/6, n=6-8) were infused scrambled or anti-miR-33 oligonucleotides (5 mg/Kg/day for 3 consecutive days), via tail vein injection. Expression of ABCA1 mRNA and protein was evaluated in the livers by real time PCR and western blot, respectively. (C) Plasma lipoprotein profiles in these same mice were obtained by fast protein liquid chromatography (FPLC) and cholesterol content of each fraction assayed by the cholesterol-oxydase method, 12 days post-infusion.

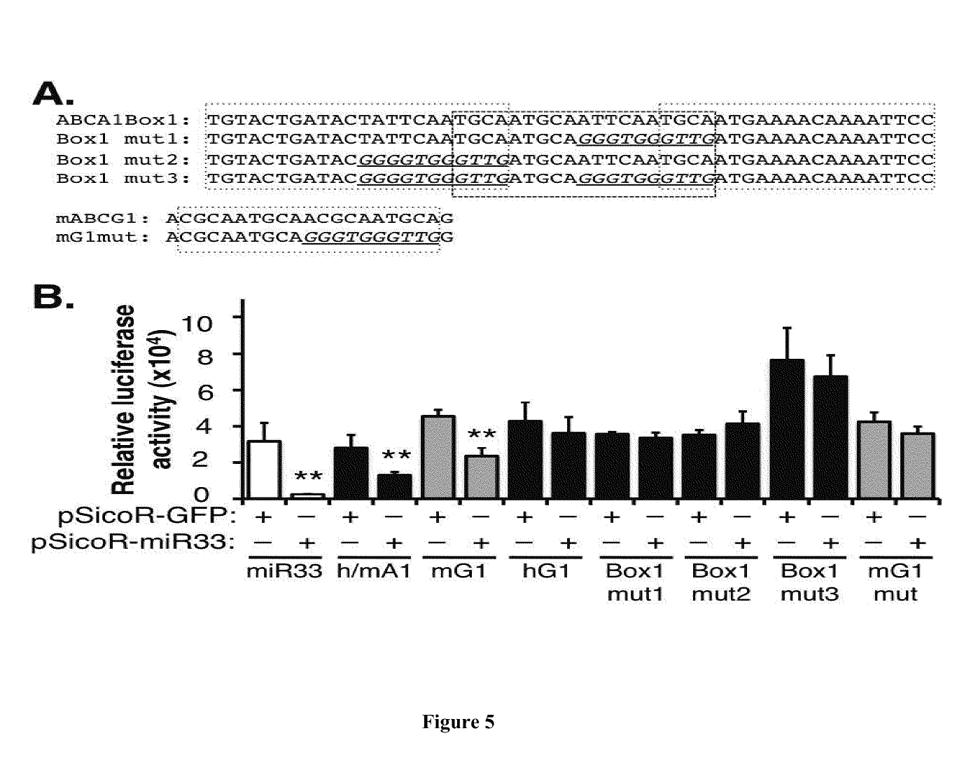

FIG. 5 shows that the mutation of specific sequences abolishes miR-33-mediated silencing. (A) Natural and mutated ABCA1 Box 1 and ABCG1 response elements for miR-33. (B) HEK293 cells were transfected as described in FIG. 4, and luciferase activity analyzed 48 h after transfection. Data are mean±SD of three independent experiments in duplicate.

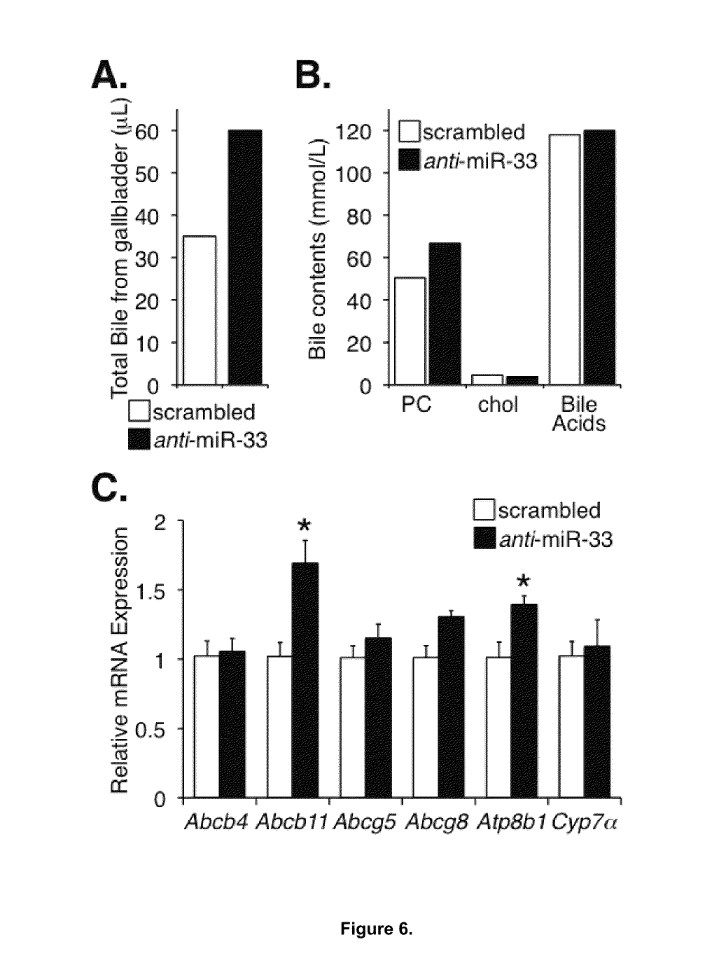

FIG. 6 shows that bile secretion is enhanced following silencing of miR-33. (A) Pooled bile recovered from the gallbladder of mice (n=5) injected with scrambled or anti-miR-33 oligonucleotides (5 mpk, i.v.) for 2 consecutive days. Mice were then kept for 7 days and fasted overnight before sample collection. (B) Levels of phosphatidylcholine (PC), cholesterol (chol) and bile acids present in pooled bile. (C) Relative expression of hepatic canalicular transporters in mice (n=5) following silencing of miR-33. Data are shown as mean±SD. *P<0.05 (unpaired T-test).

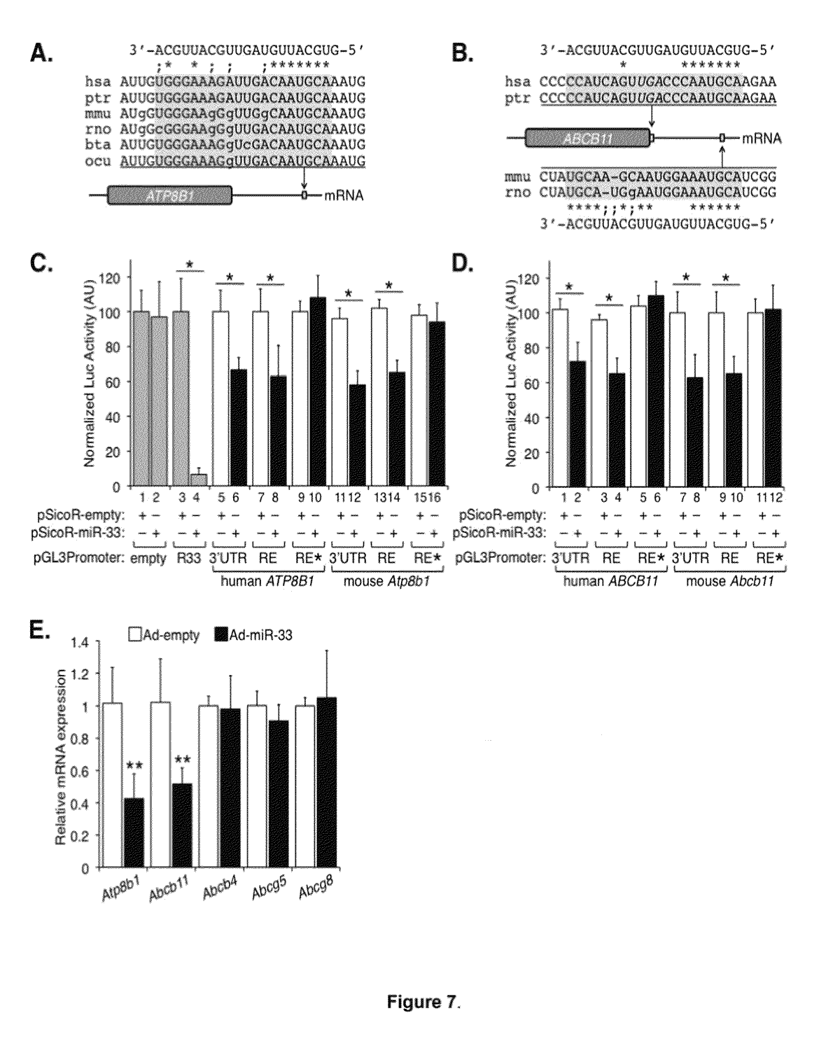

FIG. 7 shows functional miR-33 responsive elements in the 3′UTR of ATP8B1 and ABCB11. (A, B) Conserved sequences in the 3′UTR of ATP8B1 and ABCB11 are partially complementary to miR-33. The element in human ATP8B1 is located 1877-1897 nt after the stop codon. In the case of ABCB11, this element overlaps the stop codon in humans and chimps, while mice and rats show a conserved sequence 732-751 nt after the stop codon. Interestingly, other rodents such as guinea pig have both the proximal and distal miR-3 sequences in Abcb11 (data not shown). (C, D) Luciferase assays in HEK293 cells using the whole 3′UTR of human or murine ATP8B1 and ABCB11, or the isolated responsive elements (RE) identified above, or mutant responsive elements (RE*), confirm that these sequences are functional miR-33 response elements. (E) Relative expression of canalicular transporters in primary murine hepatocytes (n=3 dishes/condition) transduced 48 h with empty or miR-33 adenovirus vector. Data are mean±S.D.; **P<0.01 (unpaired T-test).

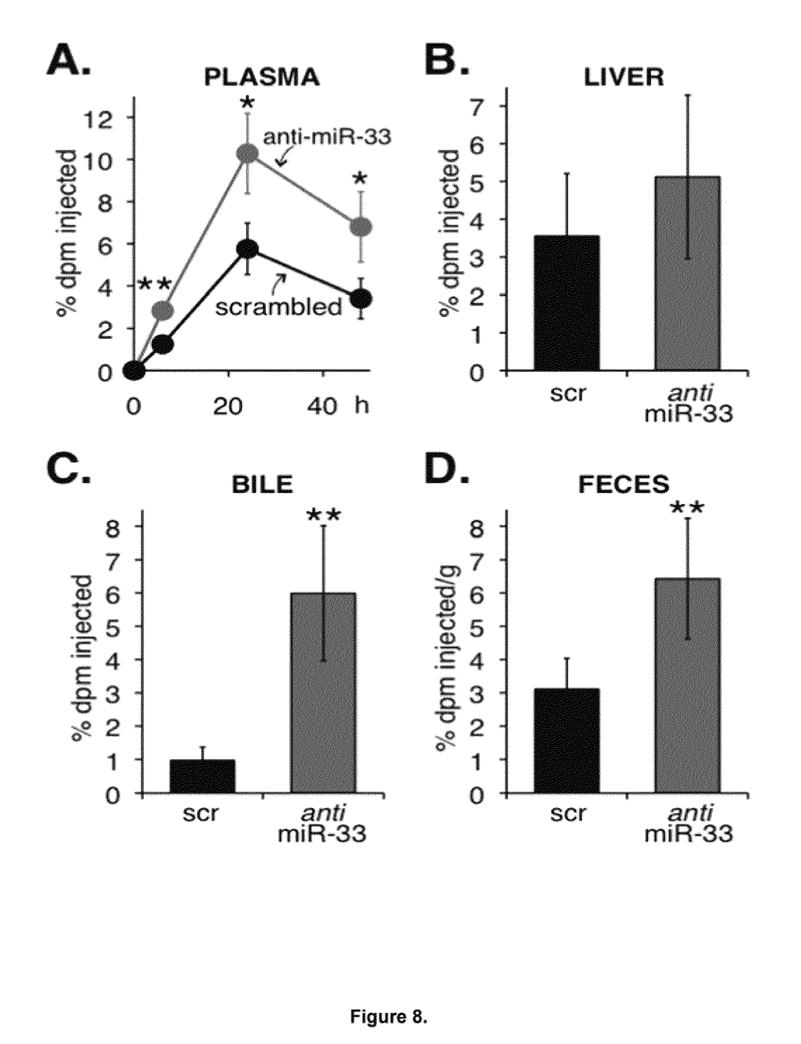

FIG. 8 shows that reverse cholesterol transport is enhanced after systemic miR-33 silencing. (A) Percentage of total injected dpm in the plasma of mice treated with scrambled or anti-miR-33 oligonucleotides (5 mpk, twice a week for 2 weeks), at 6, 24 at 48 h post-injection of [3H]-cholesterol-acLDL-loaded macrophages. *P<0.05 (unpaired T-test): **P<0.01 (unpaired T-test). (B) Percentage of total injected dpm in the liver of the same mice. (C) Percentage of total injected dpm in the be recovered from the gallbladder of the same mice. **P<0.01 (unpaired T-test). (D) Percentage of total injected dpm in the feces of the same mice. **P<0.01 (unpaired T-test).

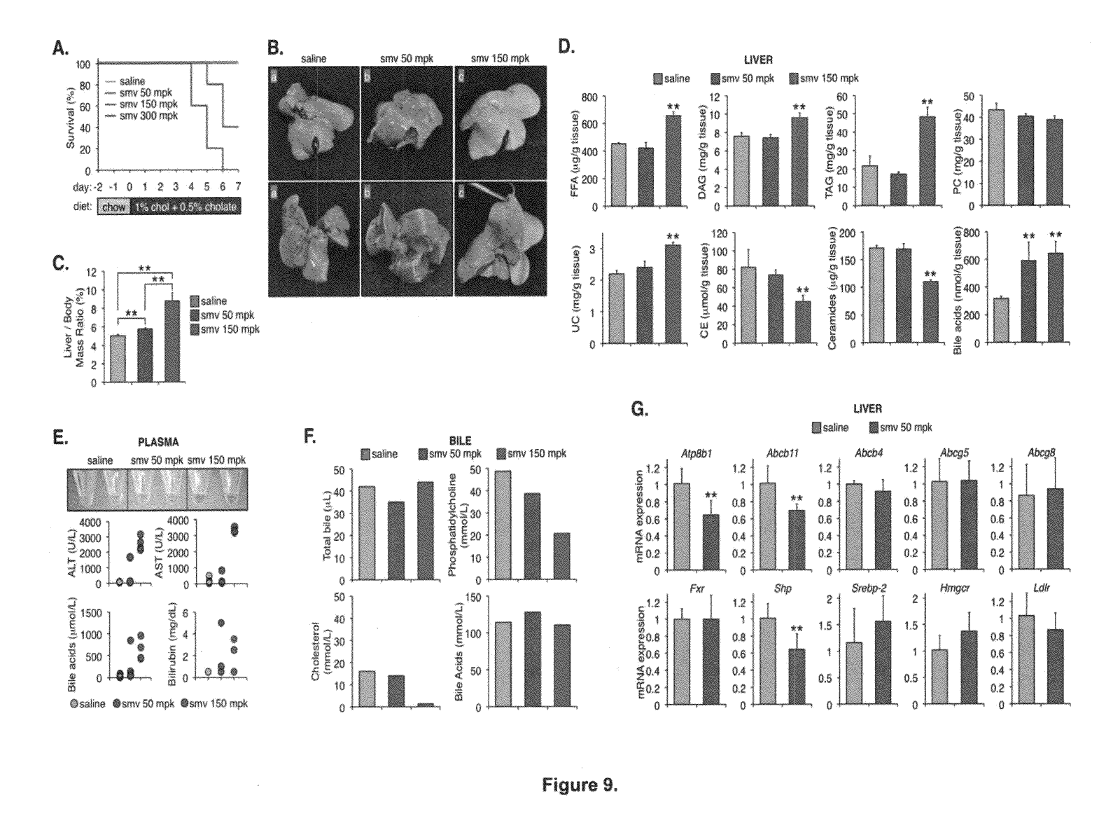

FIG. 9 shows that simvastatin and cholate Diet induce Liver Damage. Mice (n=6) were gavaged daily with 0, 50, 150 or 300 mg/Kg (mpk) simvastatin, and fed a diet containing 1% cholesterol and 0.5% cholate. Samples were collected after 7 days on the diet, or when mice appeared moribund. (A) Survival of mice is hampered by simvastatin in a dose-dependent manner. (B) Liver to total body mass ratios in the same animals. Data are shown as mean±SD. **P<0.01 (unpaired T-test). (C) Macroscopic appearance of the same livers. (O) Amounts of specific hepatic lipids as determined by ESI-MS (see Experimental Procedures), and normalized to tissue weight. **P<0.01 vs. saline (unpaired T-test). (E) Appearance of plasma, and levels of circulating alanine aminotransferase (ALT), aspartate aminotransferase (AST), bile acids and bilirubin. (F) Bile was recovered from the gallbladder, pooled, and the contents of phosphatidylcholine, cholesterol and bile acids determined with colorimetric kits (see Experimental Procedures). G Relative expression of hepatic canalicular transporters (upper panel) and other genes involved in be acid and sterol homeostasis (bottom panel) in samples from mice treated with 0 or 50 mpk simvastatin. Data are shown as mean±SD. **P<0.01 (unpaired T-test).

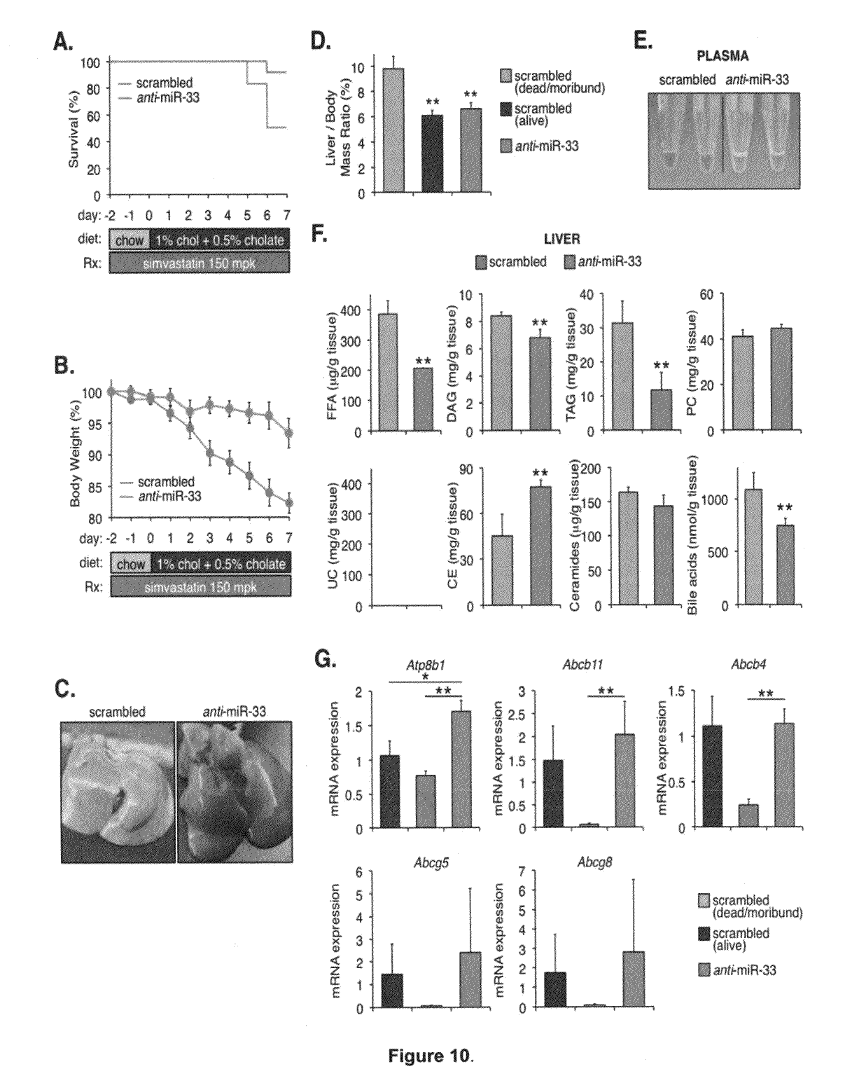

FIG. 10 shows that silencing miR-33 Rescues the Liver Damage induced by Simvastatin and Cholate Diet. Mice (n=10) were injected i.v. with scrambled or anti-miR-33 oligonucleotides (5 mpk) for two consecutive days, and then gavaged daily with 150 mg/Kg (mpk) simvastatin, and fed a diet containing 1% cholesterol and 0.5% cholate. Samples were collected after 7 days on the diet, or when mice appeared moribund. (A) Survival of mice is rescued by silencing miR-33. (B) Percentage of body weight, compared to the initial mass of each animal. Data are shown as mean±SD. (C) Macroscopic appearance of livers. (D) Liver to total body mass ratios. Data are shown as mean±SD. **P<0.01 vs. mice injected with scrambled oligos that succumbed (unpaired T-test). (E) Appearance of plasma. (F) Amounts of specific hepatic lipids as determined by ESI-MS, and normalized to tissue weight. **P<0.01 vs. scrambled (unpaired T-test). (G) Relative expression of hepatic canalicular transporters (upper panel) and other genes involved in bile acid and sterol homeostasis (bottom panel). Data are shown as mean±SD. **P<0.01 vs. mice injected with scrambled oligos that succumbed (unpaired T-test).

### DETAILED DESCRIPTION OF THE INVENTION

Cholesterol is transported through the blood bound to various lipoproteins. An important determinant of cardiovascular health is the ratio of cholesterol transported bound with high density lipoproteins (HDL) to cholesterol transported bound with low density lipoproteins (LDL). A high HDL/LDL ratio is associated with improved cardiovascular health. The goal of many therapies including the use of statins is to increase the HDL/LDL ratio by decreasing levels of LDL-cholesterol. Benefits may also be gained from an increased HDL/LDL ratio brought about by raising levels of HDL-cholesterol. Circulating levels of HDL and LDL bound cholesterol are controlled by antagonistic regulatory pathways. Expression of proteins that make up these pathways are under the transcriptional control of Sterol Regulatory Element Binding Protein-2 (SREBP-2) and the Liver X Receptor (LXR). SREBP-2 regulates the expression of proteins which facilitate cellular up take of cholesterol, and ultimately decrease levels of LDL-cholesterol in the blood. LXR regulates the expression of proteins in a pathway which ultimately increases HDL-cholesterol in the blood. Two proteins under transcriptional control of LXR are ABCA1 and ABCG1 which facilitate cellular efflux of cholesterol. ABCA1 is important for HDL formation in vivo. ABCA1 is also a transporter for lipidation of ApoAI, which is an important initial step in HDL formation. ABCG1 has been implicated in lipidation of HDL in vitro. The physiological effect of increased ABCA1 activity is increased blood HDL-cholesterol and, consequently, an increased HDL/LDL ratio.

For patients at risk for heart disease, especially patients with hypercholesterolemia, it is desirable to prevent atherosclerosis and its complications by increasing the ratio of HDL/LDL cholesterol. This may be accomplished by decreasing LDL-cholesterol and/or increasing HDL-cholesterol. Statin drugs, by way of example, atorvastatin, cerivastatin, fluvastatin, lovastatin, mevastatin, pitavastatin, pravastatin, rosuvastatin, and simvastatin will decrease LDL-cholesterol, but have little effect on HDL-cholesterol.

Disclosed are methods and compositions for increasing HDL-cholesterol in the blood by increasing expression of ABCA1. More specifically, the invention is related to oligonucleotide sequences that block the miR-33 negative control on expression of ABCA1, thereby allowing increased expression of ABCA1 and increased HDL-cholesterol levels in the blood.

In addition, there are often undesirable secondary effects induced by the administration of statins, including but not limited to: rhabdomyolysis (muscle aches, tenderness or weaknesses), cholestasis (gallstones), including Benign Recurrent Intrahepatic Cholestasis (BRIC), diarrhea, abdominal pain, dizziness, nausea, vomiting, headache, difficulty sleeping, flushing of the skin, and memory loss. It would be desirable to treat patients so as to eliminate or reduce these undesirable secondary effects. Statins have been reported not only to increase SREBP-2 expression but also to increase miR-33 (Ho, at al., (2011) FASEB J. vol. 25 no. 5 1758-1766; Marquart, et al., (2010) Proc Natl Acad Sci USA 107, 12228-12232; Najafi-Shoushtari, et al., (2010) Science. Vol. 328 no. 5985 pp. 1566-1569; Rayner, et al., (2010) Science. Vol. 328 no. 5985 pp. 1570-1573) Therefore, the inventor reasoned that miR-33 may mediate, at least in part, statin induced secondary effects and furthermore, that anti-miR-33 oligonucleotides may be beneficial in preventing or treating such effects.

Disclosed are methods and compositions for treating statin induced secondary effects by increasing expression of ABCB11 and ATP8B1. More specifically, one embodiment of the invention is related to oligonucleotide sequences that block the miR-33 negative control on expression of ABCB11 and ATP8B1 thereby allowing increased expression of ABCB11 and ATP8B1 and alleviating statin induced secondary effects. Disclosed is a method of treatment for cholestasis, as one example of treating a statin induced secondary effect by inhibiting miR-33. Also disclosed is a method for treating statin induced Benign Recurrent Intrahepatic Cholestaisis (BRIC) as another example of treating a statin induced secondary effect by inhibiting miR-33. More specifically disclosed are methods and compositions for the treatment of cholestasis with an anti-miR-33 oligonucleotide.

In addition, methods and compositions are disclosed for treating BRIC that is not associated with statins by inhibiting miR-33. Also disclosed are methods and compositions for increasing reverse cholesterol transport (ROT) in a subject by inhibiting miR-33. Increased ROT results in improved cardiovascular heath inducing but not limited to, increased HDL-cholesterol levels in blood, increased HDL uptake in liver, increased sterol (including bile acids and/or cholesterol and/or oxysterols) secretion to bile, and increased excretion of sterols through feces. It is believed that accelerated removal of cholesterol through the feces reduces the risk of cardiovascular disease, by limiting the amount of lipids that accumulate in the arteries or other tissues.

It has been thought that transcriptional control of cholesterol homeostasis by SREBP-2 and LXR was regulated through independent pathways. However, the Inventor has discovered a microRNA, designated miR-33, that is co-transcribed with SREBP-2 and controls critical aspects of cholesterol homeostasis, namely the repression of the LXR target gene ABCA1 (FIG. 1). The Inventor has also discovered target sequences on ABCA1, ABCG1, ABCB11, and ATP8B1 mRNA, through which miR-33 exerts its control. The relevance of this to humans and other animals is evident in the finding that these sequences are conserved across multiple animal species (FIG. 2B). Expression of both SREBP-2 mRNA and miR-33 are regulated by the same metabolic and steroli stimuli. Cells in high a cholesterol environment expressed low levels of miR-33 and consequently high levels of ABCA1 Whereas cells maintained in a low cholesterol environment expressed high levels of miR-33 and low levels of ABCA1. The Inventor has identified an inverse relationship between miR-33 and ABCA1, as well as miR-33 and HDL-cholesterol. When levels of mi-R-33 were increased in a subject, levels of ABCA1 and HDL-cholesterol decreased (see Examples). This inverse relationship between miR-33 and ABCA1, or miR-33 and HDL-cholesterol may be exploited to increase HDL-cholesterol and the HDL/LDL ratio in the blood. To this means the Inventor discloses antisense compounds that may be used to decrease functional levels of miR-33 in a subject, which will subsequently increase expression of ABCA1, and increase HDL cholesterol in the blood. In addition, the inventor discloses a method of treating secondary effects induced by the administration of statins. One example of a statin induced secondary effect is cholestasis. Cholestasis is a condition where bile cannot flow from the liver to the duodenum. Bile is a complex mixture of sterols (bile acids and cholesterol), phospholipids, proteins, and other organic molecules and ions that serves two main purposes: the solubilization of dietary lipids in the intestine, and the removal of waste metabolites through the feces. One symptom of cholestasis is pruritus (itchiness) which is thought to be due to interactions of serum bile acids with the opioidergic nerves. Other symptoms include jaundice (yellow color of skin and sclera), steatorrhea (malabsoption of lipids resulting in pale or even white stools), abdominal pain, nausea, vomiting. The impairment in bile secretion and/or flow results in cholestasis, which leads to hepatic injury and inflammation and, in the most severe cases, organ failure that requires liver transplantation. Primary bile is normally secreted through the apical or canalicular membrane of hepatocytes by the combined action of three distinct transmembrane transporters: ABCB11 (also known as BSEP), which facilitates the secretion of bile salts; ABCG5/ABCG8, an obligate heterodimer that facilitates cholesterol efflux; and ABCB4 (also known as MDR3/MDR2 in humans and mice, respectively) which pumps phospholipids, mostly phosphatidylcholine across the membrane. (for review see Esteller, 2008). A fourth transporter, ATP8B1, has been proposed to limit the desorption of intracellular cholesterol into the canalicular space (Paulusma et al., (2006) Hepatology 44, 195-204), perhaps by altering the symmetry of phosphatidylserine in the canalicular membrane (Paulusma et al., (2008) Hepatology 47, 268-278; Ujhazy et al., (2001) Hepatology 34, 768-775). Inactivating mutations in human ATP8B1, ABCB11 or ABCB4 result in Progressive Familial Intrahepatic Cholestasis (PFIC) type 1, 2, or 3, respectively. Accordingly, these genes are also known as FIC-1, -2, and -3, respectively. Patients with Benign Recurrent Intrahepatic Cholestasis (BRIC) also have mutations in any of the latter genes, but presumably the residual activity of the mutant transporter is sufficient to prevent the full PFIC phenotype. Loss-of-function mutations in human ABCG5 or ABCG8 result in sitosterolemia or hyperabsorption and decreased biliary excretion of dietary plant sterols (Hubacek et al., (2001 Hum Mutat 18, 359-360); Yu et at, (2002) Proc Natl Acad Sci USA 99, 16237-16242), but not in cholestasis. Transgenic knock-out mice for all these different transporters have been characterized by several independent laboratories, showing that they phenocopy the human cholestatic or sitosterolemia syndromes (Pawlikowska et al., (2004) Hum Mol Genet. 13, 881-892.; Shah at al., (2010 PLoS One 5, e8984); Wang at, 2003 Hepatology 38, 1489-1499; Yu et al., (2002) Proc Natl Acad Sci USA 99, 16237-16242). Nevertheless, both PFIC and BRIC are thought to develop as a result of the accumulation of bile containing supersaturated levels of cholesterol due to inadequate levels of bile salts (ABCB11 defect) or phospholipids (ABCB4 defect), or as a result of excess excretion of cellular cholesterol into the bile (ATP8B1 defect). The inventor has made the surprising discovery that the gallbladders of mice injected with anti-miR-33 oligonucleotides showed increased bile and that mRNA levels of both Abcb11 and Atp8b1 were significantly increased in the liver (see Example 6) suggesting that miR-33 controls bile secretion by altering the expression of Abcb11 and Atp8b1. The inventor also discloses that specific sequences within the Abcb11 and Atp8b1 genes are partially complementary to miR-33, that Abcb11 and Atp8b1 are direct targets of miR-33, and that expression of Abcb11 and Atp8b1 is significantly reduced in cells following overexpression of miR-33 (Example 7)

Statins not only increase SREBP-2 expression but also increase miR-33 (Ho, et al., (2011) FASEB J. vol. 25 no. 5 1758-1766; Marquart, et al., (2010) Proc Natl Acad Sci USA 107, 12228-12232; Najafi-Shoushtari, et al., (2010) Science. Vol. 328 no. 5985 pp. 1566-1569; Rayner, et al., (2010) Science. Vol. 328 no. 5985 pp. 1570-1573). The inventor reasoned that statins may increase the risk of cholestasis by increasing miR-33, thereby repressing the expression of both ABCB11 and ATP8B1

Similar to the relationship between miR-33 and ABCA1, an inverse relationship exists between miR-33 and ABCB11 and ATP8B1. An increase in miR-33 results in a decrease in ABCB11 and ATP8B1 and an increase in cholestasis. By administering an anti-miR-33 oligonucleotide, cholestasis may be treated or the risk of cholestasis may be reduced. In addition, the inventor has discovered that reverse cholesterol transport (ROT) may be increased in subjects receiving treatment with antisense compounds complementary to miR-33 (see example 10). Administration of an anti-miR-33 increased HDL-cholesterol levels in blood, increased HDL uptake in liver, increased sterol secretion to bile, increased excretion of sterols through feces and sterol secretion including bile acids and/or cholesterol and/or oxysterols. These are factors known to improve cardiovascular health. Therefore one embodiment of the invention is improved cardiovascular health (i.e., decreased risk of myocardial infarct, stroke and/or peripheral artery disease) by increased RCT by administration of antisense compounds complementary to miR-33.

### I. Micro RNAs

miRNAs are small, non-coding 20-24 nt RNAs that promote the silencing of their target genes by binding to specific, partially complementary regions in the 3′ untranslated regions (UTR) of the target mRNA. This results in RNA interference and/or translational repression of the target gene (for review see Bartel, (2009) Cell; 136:215; Olena, and Patton (2010) J Cell Physiol.; 222:540). miRNAs can be transcribed from their own promoter or may be encoded in the introns of other genes. It is speculated that in the later case these miRNA might be expressed when the “hosting” mRNA is transcribed. Regardless, micro RNAs are transcribed as primary Pri-miRNA (200-400 nt) which are first processed by the exonuclease Drosha resulting in a Pre-miRNA (100-150 nt), which is then exported to the cytoplasm via exportin, a nuclear export factor. Pre-miRNA is then further cleaved by Dicer, a ribonuclease III and its cofactors (PACT and TRBP) to generate a mature miRNA (20-24 nt) (Bartel, (2009) Cell; 136:215; Olena, and Patton, (2010) J Cell Physiol.; 222:540) which contains duplexes of 19 to 25 nucleotides. The double-stranded RNA dissociates and one strand is incorporated into the RNA-induced silencing complex (RISC). The miRNA/RISC complex is then capable of binding to target mRNAs and inhibiting expression through cleavage and degradation of the target mRNA (RNA silencing) and/or by interfering with translation.

The present invention relates to a microRNA that is co-expressed with SREBP-2 mRNA and has a silencing effect on ABCA1, ABCB11 and ATP8B1. Specifically, the Inventor has identified a microRNA, designated miR-33, encoded within intron 16 of human of SREBP-2 (FIG. 2A). A single intact mRNA is transcribed, and processing through post-transcriptional modification mechanisms to produce both SREBP-2 mRNA and miR-33. miR-33 is free to bind to the 3′-UTR of ABCA1 mRNA and reduce levels of ABCA1 expressed, ultimately decreasing levels of HDL-cholesterol in the blood. The present invention also relates to antisense compounds, including antisense oligonucleotides that are complementarity with miR-33, or its precursors, and which interfere with miR-33 mediated silencing of ABCA1. The administration of antisense compounds, including antisense oligonucleotides, that are complementary to miR-33, will result in increase levels of ABCA1, and consequentiality increase HDL-cholesterol in the blood. In addition, the administration of antisense compounds, including antisense oligonucleotides, that are complementary to miR-33, will result in increased ABCB11 and ATP8B1, and reduce or alleviate the secondary effects induced by the administration of statins.

The inventor has identified the specific sequences in the 3′-UTR of ABCA1 and ABCG1 mRNA, which are targets for miR-33-mediated silencing. Specifically, in human ABCA1, the inventors identified a proximal element, nucleotides 120-172 downstream of the stop codon; 5′-UGUACUGAUACUAUUCAAUGCAAUGCAAUUCAAUGCAAUGAAAACAAAAUUCCA-3′) (SEQ ID NO: 1), and have termed this Box 1 (FIGS. 3A and C), that contains three overlapping miR-33 responsive elements, and a distal element at nucleotides 1,465-1,481 after the stop codon; 5-UUAAUUGCAACAAUGCAG-3′) (SEQ ID NO: 2), termed Box 2 (FIGS. 3A and C). For human ABCG1, a single putative miR-33 response element was identified at nucleotides 516-541; 5′-UGCAAGCCAAAAGUCGAUCAAUCGCAU-3′) (SEQ ID NO: 3), after the stop codon (FIGS. 3B and C). The same putative sequences are found in nucleotides 120-171 and 1,426-1,442, and 715-733 in the mouse ABCA1 and ABCG1 genes, respectively. Importantly, these sequences are evolutionarily conserved across animal species (FIG. 3A, B).

### II. Antisense Compounds

A number of RNA interference devices may be employed to modulate miR-33 suppression of ABCA1 and plasma HDL-cholesterol levels, or ABCB11 and ATP8B1 and statin induced secondary effects. RNA interfering systems that include RNA or DNA oligonucleotides that are complementary to a target nucleic acid and are commonly referred to as “antisense” oligonucleotides. Antisense oligonucleotides will be complementary to a chosen target nucleic acid so that the antisense oligonucleotide will specifically hybridize to the target nucleic acid. An antisense therapy requires first identifying a target nucleic acid sequence whose function is to be modulated. In the present invention, the target nucleic acid whose function is to be modulated is miR-33, or miR-33 precursors including but not limited to miR-33 pri-miRNA, and miR-33 pre-miRNA. Binding to miR-33, or interfering with any step in transcription or post-transcriptional processing of miR-33 precursors will result in reduced levels of miR-33 available for suppression of ABCA1. The present invention encompasses antisense oligonucleotides that are complementary to the corresponding target nucleic acids sequences for miR-33 (NC_{—}000022.10, reference coordinates 42,296,953 42,296,973), and its precursor (NC_{—}000022.10, ref. coordinates 42,296,948-42,297,016). The present invention encompasses antisense oligonucleotides including but not limited to the following.

An antisense oligonucleotide targeted to miR-33 may be complementary in whole or in part to 5′-GUGCAUUGUAGUUGCAUUGCA-3′ (SEQ ID NO: 4), a nucleic acid sequence encoding miR-33. One more preferred example of an antisense oligonucleotide targeted to miR-33 is 5′-TGCAATGCAACTACAATGCAC-3′ (SEQ ID NO: 5), which is complementary to the nucleic acid sequence encoding miR-33. Other preferred examples include any 8, 9, 10, 11, 12, 13, 14, 15, 16, 17, 18, 19, or contiguous nucleotide-bases set forth in the sequence 5″-TGCAATGCAACTACAATGCAC-3″ (SEQ ID NO: 5). An antisense oligonucleotide targeted to miR-33 preRNA may be complementary in whole or in part to 5′-CUGUGGUGCAUUGUAGUUGCAUUGCAUGUUCUGGUGGUACCCAUGCAAUGUUU CCACAGUGCAUCACAG-3′ (SEQ ID NO: 6), a nucleic acid sequence encoding miR-33 preRNA. One most preferred example of an antisense oligonucleotide that is complementary to the target nucleic acid miR-33 preRNA is 5′-CTGTGATGCACTGTGGAAACATTGCATGGGTACCACCAGAACATGCAATGCAACTA CAATGCACCACAG-3′ (SEQ ID NO: 7), which is complementary to the nucleic acid sequence encoding miR-33 preRNA. Other preferred examples include any 8, 9, 10, 11, 12, 13, 14, 15, 16, 17, 18, 19, 20, or 21 contiguous nucleotide-bases set forth in the sequence 5″-CTGTGATGCACTGTGGAAACATTGCATGGGTACCACCAGAACATGCAATGCAACTA CAATGCACCACAG-3″ (SEQ ID NO: 7). An antisense oligonucleotide that is complementary to the target nucleic acid miR-33 preRNA may also include more than 21 contiguous nucleotide-bases set forth in the sequence 5″-CTGTGATGCACTGTGGAAACATTGCATGGGTACCACCAGAACATGCAATGCAACTA CAATGCACCACAG-3″ (SEQ ID NO: 7). A most preferred example of an antisense oligonucleotide targeted to both miR-33, and miR-33 preRNA, and is 5-TGCAACTACAATGCA-3′ (SEQ ID NO: 8). It is appreciated that antisense oligonucleotides may substitute uracil (U) with thymine (T), or thymine (T) with uracil (U) and maintain complementation to the target nucleic acid.

To be effective it is not necessary for an antisense oligonucleotide to hybridization 100 percent with the target nucleic acid. Antisense oligonucleotides are chosen which are sufficiently complementary to the target nucleic acids, and which bind sufficiently well and with sufficient specificity, to give the desired reduction in effective levels of mi-R33. The target nucleic adds of the present invention include miR-33, miR-33 pri-miRNA, and miR-33 pre-miRNA. It is expected that antisense oligonucleotides which are complementary to the entire sequence of one or more of these target nucleic acids will be effective in reducing functional levels of miR-33. It is also expected that antisense oligonucleotides which are complementary to less than the entire sequence of one or more of the target nucleic acids will be effective in reducing functional levels of miR-33. Antisense oligonucleotides effective in reducing effective levels of miR-33 are expected to be complementary to at least 8, preferably at least 10, more preferably at least 12; more preferably to at least 14; even more preferably to at least 18; yet more preferably to at least 22 nucleic acids of one or more of the target nucleic acids. It is also preferred that the antisense compound hybridize to nucleic acids that are contiguous.

An inverse relationship exists between levels of miR-33, HDL-lipidation in vitro, and plasma HDL-cholesterol in vivo. This is demonstrated in the examples by the administration of an oligonucleotide encoding miR-33, via a viral vector. In summary administration of the viral vector caused increased levels of miR-33, increased suppression of ABCA1, and decreased plasma HDL-cholesterol. One of ordinary skill in the art will appreciate that this inverse relationship between miR-33 and HDL-cholesterol may be exploited to increase plasma HDL-cholesterol levels, and that through administration of the antisense compounds described above, functional levels of miR-33 will be reduced, expression of ABCA1 will increase, and plasma HDL-cholesterol will increase. The physiological effect will be an increased HDL/LDL ratio and improved cardiovascular health.

As used herein, the term “antisense compound” is meant to include, antisense oligonucleotides, with or without modified backbones, and is intended to include other chemical compounds that specifically bind to the same targeted nucleic acids that are described herein, and that provide the same regulatory effect on miR-33 or ABCA1 expression as the subject antisense oligonucleotides.

As used herein, the term “anti-miR-33” oligonucleotide is meant to include antisense compounds or antisense oligonucleotides that bind to miR-33 or a precursor of miR-33 in part or in whole.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′,” which may also be expressed as “5-A-C-T-3′.” The Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second noncomplementary target.

The term “hybridization”, as used herein, means hydrogen bonding, which may be Watson-Crick, Hoogsteen or reversed Hoogsteen hydrogen bonding, between complementary nucleotide bases. For example, adenine and thiamine, and guanine and cytosine, respectively, are complementary nucleobases that pair through the formation of hydrogen bonds. “Complementary”, as that term is used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are complementary to each other at that position. The igonucleotide and the DNA or RNA are complementary to each other when a sufficient number of corresponding positions in each molecule are occupied by nucleotides that can hydrogen bond with each other. “Specifically hybridize” means that a particular sequence has a sufficient degree of complementarity or precise pairing with a DNA or RNA target sequence that stable and specific binding occurs between the oligonucleotide and the DNA or RNA target. It is understood in the art that the sequence of an antisense compound need not be 100% complementary to that of its target nucleic acid to be specifically hybridizable. Typically, for specific hybridization in vitro, moderate stringency conditions are used such that hybridization occurs between substantially similar nucleic adds, but not between dissimilar nucleic adds. In in vitro systems, stringency conditions are dependent upon time, temperature and salt concentration as can be readily determined by the skilled artisan. (See, e.g., Sambrook et al., Molecular Cloning: A Laboratory Manual, Cold Spring Harbor Laboratory Press, NY (1989)). For in vivo antisense methods, the hybridization conditions consist of intracellular conditions which govern the hybridization of the antisense oligonucleotide with the target sequence. An antisense compound specifically hybridizes to the target sequence when binding of the compound to the target DNA or RNA molecule interferes with the normal translation of the target DNA or RNA such that a functional gene product is not produced, and there is a sufficient degree of complementarity to avoid non-specific binding.

### A. Modified Oligonucleotide Backbones

While antisense oligonucleotides comprised of DNA, or RNA are a preferred form of antisense compound, the present invention contemplates other oligomeric antisense compounds, including, but not limited to, locked nucleic add (LNA) oligonucleotides. Examples of LNA included polynucleotides whereby the ribose moiety of the nucleotide is modified by forming a bridge connecting the 2′ oxygen and 4′ carbon. In addition, there are oligonucleotide mimetics containing modified backbones (which may be referred to herein as “modified internucleoside linkages”). As defined herein, oligonucleotides having modified backbones include those that retain a phosphorous atom in the backbone, as well as those that do not have a phosphorous atom in the backbone. Modified oligonucleotide backbones which are useful in the subject antisense oligonucleotides include, for example, phosphorothioates, chiral phosphorothioates, phosphorodithioates, phosphotriesters, aminoalkylkphosphotriesters, methyl and other alkyl phosphonates including 3′-alkylene phosphonates and chiral phosphonates, phosphinates, phosphoramidates including 3′ aminophosphoramidate and aminoalkylphosphoramidates, thionophosphoramidates, thionoalkylphosphonates, and boranophosphonates having normal 3′-5′ linkages, linked analogs of these, and those having inverted polarity wherein the adjacent pairs of nucleoside units are linked 3′-5′ to 5′-3′ or 2′-5′ to 5-2′.

Various salts, mixed salts and free acid forms are also included. References that teach the preparation of such modified backbone oligonucleotides are provided, for example, in U.S. Pat. No. 5,945,290. Modified oligonucleotide backbones that do not include a phosphorous atom therein may comprise short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; alkenecontaining backbones; sulfamate backbones; methyleneimino and methylenehydrazino backbones; sulfonate and sulfonamide backbones; amide backbones; and others having mixed N, 0, S and CH₂ component parts. References that teach the preparation of the oligonucleotides listed above are provided in U.S. Pat. No. 5,945,290.

Other useful oligonucleotide mimetics, which are useful in the subject antisense oligonucleotides, comprise replacement of both the sugar and the internucleoside linkage—i.e., the backbone-of the nucleotide units with novel groups. One such oligomeric compound that has excellent hybridization properties is a peptide nucleic acid. See, e.g., Nielsen et al., Science, 254:1497-1500 (1991); and U.S. Pat. Nos. 5,539,082; 5,714,331; and 5,719,262. In such peptide nucleic acid compounds the sugar-backbone of an oligonucleotide is replaced with an amide containing backbone, in particular with an aminoethylglycine backbone. The nucleobases are retained and are bound directly or indirectly to aza nitrogen atoms of the amide portion of the backbone.

Other useful modified oligonucleotides are those having phosphorothioate backbones and oligonucleotides with heteroatom backbones, and in particular —CH₂—NH-0-CH₂—, —CH₂—N(CH₃)-0-CH₂—, —CH₂-0-N(CH₃)—CH₂—, —CH₂—N(CH₃)—N(CH₃)—CH₂—, and -0-N(CH₃)—CH₂—CH₂—, wherein the native phosphodiester backbone is represented as —O—P—O—CH₂—, (as disclosed in U.S. Pat. No. 5,489,677), and the amide backbones disclosed in U.S. Pat. No. 5,602,240. Also useful are oligonucleotides having morpholino backbone structures as taught in U.S. Pat. No. 5,304,506.

Modified oligonucleotides can also contain one or more substituted sugar moieties (which may be referred to herein as “modified sugar moieties”). Useful oligonucleotides comprise one of the following at the 2′ position: OH; F; 0-, S-, N-alkyl; N-alkenyl; N-alkynyl; or O-alkyl-O-alkyl, wherein the alkyl, alkenyl, or alkynyl may be substituted or unsubstituted C1 to C10 alkyl, or C2 to C10 alkenyl and alkynyl; 0(CH₂)0(CH₃); 0(CH₂)0(CH₂)ₙCH₃: 0(CH₂)nNH₂; or 0(CH₂)ₙCH₃ (where n=1 to 10); Cl; Br; CNB; CF₃; OCF₃; NO₂; N₃; NH₂; heterocycloalkyl; heterocycloalkaryl; aminoalkylamino: polyalkylamino; substituted silyl; an RNA cleaving group: a cholesterol group; a reporter group; an intercalator; a group for improving the pharmacokinetic properties of an oligonucleotide; or a group for improving other substituents having similar properties. Oligonucleotides can also have sugar mimetics such as cyclobutyls in place of the pentafuranosyl group. A preferred modified sugar moiety is a 2′-0-methoxyethyl sugar moiety.

Other useful antisense compounds may include at least one nucleobase modification or substitution. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine, 5-hydroxymethylcytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocystine, 5-halouracil and cytosine, 5-propynyl uracil and cytosine, 6-azo uracil, cytosine and thymine, 5-uracil, 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substitutes uracils and cytosines, 7-methylguanine and 7-methyladenine, 8-azaguanine and 8-azaadenine, 7-deazaguanine and 7-deazaadenine and 3-deazaguanine and 3-deazaadenine.

The antisense compounds of the present invention may be conveniently and routinely made through the well-known technique of solid phase synthesis. Equipment for such synthesis is available from several manufacturers and vendors including, for example; Applied Biosystems; Foster City, Calif. Any other means for such synthesis known in the art may additionally or alternatively be employed. It is also well known to use similar techniques to prepare modified oligonucleotides such as the phosphorothionates and alkylated derivatives that are discussed above. Where appropriate, the antisense compounds of the present invention including antisense oligonucleotides may also be made through recombinate molecular biology methods know in the art.

### B. Formulations

A “pharmaceutically acceptable carrier” is a pharmaceutically acceptable solvent, suspending agent or any other pharmacologically inert vehicle for delivering one or more of the subject antisense oligonucleotides to an vertebrate. The pharmaceutically acceptable carrier may be a liquid or a solid and is selected with the planned manner of administration in mind so as to provide for the desired bulk, consistency, and other pertinent transport and chemical properties, when combined with one or more of the subject antisense oligonucleotides and any other components of a given pharmaceutical composition. Typical pharmaceutically acceptable carriers include, but are not limited to, saline solution: binding agents (e.g., pregelatinized corn starch, polyvinylpyrrolidone or hydroxypropyl methylcellulose, or etc.); fillers (e.g., lactose and other sugars, microcrystalline cellulose, pectin, gelatin, calcium sulfate, ethyl cellulose, polyacrylates or calcium hydrogen phosphate, and the like); lubricants (e.g., magnesium stearate, starch, polyethylene glycol, sodium benzoate, sodium acetate, and the like); disintegrates (e.g., starch, sodium starch glycolate, and the like); or wetting agents (e.g.; sodium lauryl sulfate, and the like).

The pharmaceutical compositions of this invention may be administered in a number of ways depending upon whether local or systemic treatment is desired, and upon the area to be treated. Administration may be topical (including opthalmic, vaginal, rectal, intranasal, transdermal), oral or parenteral, for example, by intravenous drip, subcutaneous, intraperitoneal or intramuscular injection or intrathecal or intraventricular administration, such as, for example, by intracerebral ventricular injection (ICV). It is believed that the subject antisense oligonucleotides can also be administered by tablet, since the toxicity of the oligonucleotides is very low. Administration can be either rapid as by injection or over a period of time as by slow infusion or administration of slow release formulations. For treating tissues in the central nervous system, administration can be by injection or infusion into the cerebrospinal fluid.

An antisense oligonucleotide can be coupled to any substance known in the art to promote uptake by a target cell or tissue such as by way of non-limiting example an antibody to the transferrin receptor, and administered by intravenous injection. The antisense compound can be linked with a viral vector, for example, which can make the antisense compound more effective and/or increase the transport of the antisense compound to target cells or tissue.

The subject antisense compounds may be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. For example, cationic lipids may be included in the formulation to facilitate oligonucleotide uptake. One such composition shown to facilitate uptake is LIPOFECTIN (available from GIBCOBRL, Bethesda, Md.).

The antisense compounds of the present invention can include pharmaceutically acceptable salts, esters, or salts of such esters, or any other compound which, upon administration to an animal, including a human, is capable of providing—directly or indirectly—the biologically active metabolite or residue thereof. Accordingly, for example, the invention is also meant to include prodrugs and pharmaceutically acceptable salts of the compounds of the invention, pharmaceutically acceptable salts of such prodrugs, and other bioequivalents.

As used herein, the term “prodrug” means a therapeutic agent that is prepared in an inactive form that is converted to an active form within the body or cells thereof by the action of endogenous enzymes or other chemicals and/or conditions. The term “pharmaceutically acceptable salts” means physiologically and pharmaceutically acceptable salts of the compounds of the invention: i.e., salts that retain the desired biological activity of the parent compound and do not impart undesired toxicological effects thereto. As applied to antisense oligonucleotides compounds, a prodrug includes an oligonucleotide that once administered to a subject is transcribed to an effective antisense oligonucleotide compound.

The present invention also includes pharmaceutical compositions and formulations that include the antisense compounds of the invention. Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water or non-aqueous media, capsules, sachets or tablets. Thickeners, flavorings, diluents, emulsifiers, dispensing aids or binders may be desirable. Formulations for parenteral administration may include sterile aqueous solutions which may also contain buffers, diluents and other suitable additives.

### III. Administration of Antisense Compounds.

An effective amount of an antisense oligonucleotide may be introduced into a subject to providing a more beneficial HDL/LDL ratio. Administration of an effective amount of an antisense oligonucleotide, that shares complementation with miR-33, pri-miR miR-33, or pre-miRNA miR-33, into a subject, will reduce functional levels of miR-33, thereby relieving repression of ABCA1 and increasing HDL-cholesterol in the blood. A skilled practitioner will determine an effective amount of antisense oligonucleotide to be administered to a subject empirically. An antisense oligonucleotide may be administered by a skilled practitioner in any formulation and by any route of administration including those disclosed herein. It is also expected that a prodrug in the form of a oligonucleotide may be administered, which when transcribed, may produce an effective antisense oligonucleotide.

The term “effective amount” is used herein to refer to the minimal amount of a pharmaceutical composition that should be administered to a mammal in order to achieve a significant therapeutic effect. The dosages will depend on many factors including the mode of administration. Typically, the amount of antisense oligonucleotide contained within a single dose will be an amount that effectively modulates the HDL/LDL ratio related undesired condition without inducing significant toxicity. In addition, or in the alternative, the amount of antisense oligonucleotide contained within a single dose will be an amount that will alleviate or modulates an statin induced secondary effect. An effective amount of the antisense oligonucleotides and compositions of the present invention will comprise an amount of antisense oligonucleotides which will cause a significant change in the HDL/LDL ratio. In addition or in the alternative, an effective amount of the antisense oligonucleotides and compositions of the present invention will comprise an amount of antisense oligonucleotides which will alleviate or modulate some of all of the symptoms of statin-induced secondary effects. In particular where the secondary effect is cholestasis, an effective amount of the antisense oligonucleotides and compositions of the present invention will comprise an amount of antisense oligonucleotides which alleviate symptoms including pruritus, jaundice, abdominal pain, steatorrhea, nausea, vomiting. The effectiveness of the treatment may be assessed by blood tests to measure the levels of liver-derived metabolites, including alanine transaminase (ALT), aspartate transaminase (AST), bilirubin (conjugated or unconjugated), bile acids, alkaline phosphatase, or gamma-glutamyl-transpeptidase. The effectiveness of the treatment may also be assessed by CT scan of the abdomen, MRI of the abdomen, ultrasound of the abdomen, or other diagnostic techniques. In addition the effectiveness of the treatment may also be assessed by questioning the subject. An effective amount for treating Benign Recurrent Intrahepatic Cholestasis is an amount that improves cholestasis, and may be measured as described above. For example, an effective amount is that which decreases pruritus and/or jaundice, and/or steatorrhea, and/or normalizes plasma levels of specific liver-derived metabolites such as bile acids and/or bilirubin and/or ALT, among others. An effective amount for increasing reverse cholesterol transport is an amount that which improves the HDL/LDL-cholesterol ratio by increasing HDL-cholesterol in circulation, and/or increases sterols in feces, and/or increases expression of genes involved in RCT (such as ABCA1) in tissues such as the liver, and/or results in regression or stabilization of atherosclerotic lesions in the wall of arteries.

The effective amount may be given daily, weekly, monthly, or fractions thereof. Typically, a pharmaceutical composition of the invention can be administered in an amount from about 0.01 mg up to about 500 mg per kg of body weight per day (e.g., 0.5 mg, 1 mg, 2 mg, 5 mg, 10 mg, 50 mg, 100 mg, or 250 mg per kilogram body weight per day). Dosages may be provided in either a single or multiple dosage regimens. For example, in some embodiments the effective amount is a dose that ranges from about 0.001 mg to about 0.1 mg, from about 0.1 mg to about 1 mg, from about 1 mg to about to mg, from about 10 mg to about 25 mg, from about 25 mg to about 100 mg, from about 100 mg to about 500 mg, from about 500 mg to about 1000 mg from about 1 gram to about 25 grams of the antisense compound per kilogram body weigh per day, or weekly, or monthly equivalents thereof.

These are simply guidelines since the actual dose must be carefully selected and titrated by the attending physician based upon clinical factors unique to each patient. The optimal daily dose will be determined by methods known in the art and will be influenced by factors such as the age of the patient and other clinically relevant factors. In addition, patients may be taking medications for other diseases or conditions. The other medications may be continued during the time that the antisense oligonucleotides are given to the patient, but it is particularly advisable in such cases to begin with low doses to determine if adverse side effects are experienced.

The antisense compounds of the instant invention may be used to treat any subject for whom modified HDL levels, improved cardiovascular health or modified statin induced secondary symptoms are desired. Since hepatic ABCA1 is an important element in the generation of plasma HDL, it is anticipated that a combination therapy that includes statins and antisense compounds directed at miR-33, will result in both decreased LDL and increased HDL levels, thus improving the prognosis for patients with hypercholesterolemia and cardiovascular disease. It is anticipated that antisense compounds of the instant invention targeted to miR-33 could be used, alone or as coadjuvants with statins or other drugs, for better management of hypercholesterolemia/dyslipidemia, and thus ameliorate atherosclerosclerosis, cardiovascular disease, stroke and peripheral artery disease. Statin drugs, by way of non-limiting example, including atorvastatin, cerivastatin, fluvastatin, lovastatin, mevastatin, pitavastatin, pravastatin, rosuvastatin, simvastatin, may be co-administered, or administered separately according the patients specific need, based upon clinical factors unique to each patient and as selected and titrated by the attending physician

In a preferred embodiment is an antisense compound complementary to the nucleic add sequence of miR-33.

In another preferred embodiment is a antisense compound complementary to the nucleic add sequence of miR-33 priRNA.

In another preferred embodiment is a antisense compound complementary to the nucleic add sequence of miR-33 preRNA.

In another preferred embodiment is a method of regulating HDL-cholesterol levels in a subject by administering to a subject an effective amount of an antisense compound complementary to miR-33.

In another preferred embodiment is a method of providing a beneficial HDL/LDL ratio in a subject by administering an antisense compound complementary to the nucleic acid sequence of miR-33 and a statin.

In another preferred embodiment is a method of providing a beneficial HDL/LDL ratio in a subject by administering an antisense compound complementary to the nucleic acid sequence of miR-33 priRNA and a statin.

In another preferred embodiment is a method of providing a beneficial HDL/LDL ratio in a subject by administering an antisense compound complementary to the nucleic acid sequence of miR-33 preRNA and a statin.

In a most preferred embodiment is a method of treating statin induced secondary effects in a patient in need, by administering an antisense compound complementary to the nucleic add sequence of miR-33.

In more preferred embodiment is a method of treating statin induced secondary effects in a patient in need by administering an antisense compound complementary to the nucleic acid sequence of miR-33 priRNA.

In another preferred embodiment is a method of treating statin induced secondary effects in a patient in need, by administering an antisense compound complementary to the nucleic acid sequence of miR-33 preRNA while continuing to administer statins.

In another preferred embodiment is a method of treating Benign Recurrent Intrahepatic Cholestasis associated with statins by administering an effective amount of an antisense compound complementary to miR-33.

In another preferred embodiment is a method of treating Benign Recurrent Intrahepatic Cholestasis not associated with statins by administering an effective amount of an antisense compound complementary to miR-33.

In yet another preferred embodiment is a method of improving cardiovascular health or treating cardiovascular disease by increasing reverse cholesterol transport (RCT) by administering an effective amount of an antisense compound complementary to miR-33.

The term “vector” as used herein, refers to vectors for the delivery of therapeutic agents. Examples include, but are not limited to, viral vectors, liposomes, large natural polymers, large synthetic polymers, and polymers comprised of both natural and synthetic components.

As used herein, “percent Identity” of two amino acid sequences or of two nucleic acids is determined using the algorithm of Karlin and Altschul (Proc. Natl. Acad. Sci. USA, 87:2264-2268, 1990), modified as in Karlin and Altschul (Proc. Natl. Acad. Sri. USA, 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12, to obtain nucleotide sequences homologous to a nucleic acid molecule of the invention. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g. XBLAST and NBLAST) are used.

The term “subject” as used herein in reference to HDL/LDL ratio, refers to any mammal in which is it desirable to modulate HDL, LDL, ABCA1, miR-33, miR-33 priRNA, miR-33 preRNA or biochemicals related to cardiovascular or hypercholesterolemic disease.

The term subject The term “subject” as used herein in reference to statin induced secondary effects, includes any mammal in which it is desirable to treat modulate miR-33 to treat or alleviate the secondary effects induced by the administration of statins.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLES

### Methods and Materials

Plasmid constructs. A fragment containing miR-33 flanked by 150 by upstream and 150 by downstream of genomic sequence was amplified by polymerase chain reaction (PCR) from mouse genomic DNA obtained from tail biopsies from C57Bl/6 mice. The reverse primer used in the PCR contained the appropriate terminator sequence for RNA-pol-III (TTTTTCT). This fragment was cloned into the HpaI-XhoI sites of pSicoR-GFP (Addgene), which provides a U6 promoter to control the expression of the transgene, thus generating pSicoR-miR33. The integrity of the clones was analyzed by sequencing.

Cells. HEK293 cells and Hep3B cells (American Type Culture Collection) were maintained in DMEM plus 10% FBS. Human monocyte-derived macrophages and mouse thioglycollate-elicited primary peritoneal macrophages were obtained and maintained as described (Lusis, (2000) Nature: 407:233). Murine macrophages were incubated for 48 h in media A (DMEM supplemented with 10% lipoprotein-deficient serum (LPDS) (Intracel), 100 μmol/L mevalonic acid (Sigma) and 50 μmol/L pravastatin (Cayman Chemicals)), in the presence or absence of 10 cholesterol (Sigma) and 1 μg/mL 25-hydroxycholesterol (Sigma). Human macrophages were incubated in media A supplemented with or without 40 μg/mL oxidized LDL (Biomedical Technologies). Where described, cells were either transfected with pSicoR-GFP or pSicor-miR33 using Lipofectamine 2000 (invitrogen) following the manufacturer's recommendations, or transduced with a CFP or a miR-33 adenovirus.

HEK293 cells and HepG2 cells (American Type Culture Collection) were maintained in DMEM supplemented with 10% FBS. Luciferase reporter constructs containing miR-33 response elements or the 3′UTR of human/mouse ATP8B1 or ABCB11 were generated by cloning each sequence into a XbaI site in pGL3 Promoter (Promega). 3′UTR fragments were amplified from human or mouse genomic DNA using Platinum Pfx (Invitrogen). Scrambled and anti-miR-33 oligonucleotides were generously provided by miRagen Therapeutics Inc. (Boulder, Colo.).

Primary Hepatocytes. Cells were isolated from 8-10 week old, male C57BL/6 mice fed regular chow, using Perfusion and Digest buffers from Invitrogen. Cells were resuspended in William's E Medium (Invitrogen) supplemented with Plating Supplements (Invitrogen), plated in 12- or 6-well BioCoat Collagen I plates (BD), and incubated at 37° C. and 5% CO₂ for 6 h. Then, the media was switched to William's E supplemented with Maintenance Supplements (Invitrogen). Where indicated, cells were transduced in Maintenance Medium with Adeno-empty or Adeno-miR-33 adenovirus (MO 3). Following 72 h after transduction, mRNA were extracted.

Cholesterol efflux assays. HEK293 were seeded in 24-well plates (0.25°105 cells/well) and transfected with scrambled or anti-miR33 oligonucleotides (Dharmacon) following the manufacturer's recommendations. After 36 h in complete media, the cells were washed and incubated for an additional 16 h hr in DMEM supplemented with 10% LPDS and [3H]-cholesterol (1 μCi/mL). After 16 h, the cells were washed with PBS, and incubated in DMEM supplemented with 0.2% BSA for a 2 h equilibration period. Where indicated, the cells were incubated with T0901317 (1 μmol/L) and 9-cis retinoic acid (1 mmol/L) during radiolabeling and equilibration. To determine cholesterol efflux, the cells were rinsed three times with PBS and then incubated for 6 h in DMEM supplemented with 0.2% BSA and, where indicated, with either ApoAI (15 μg/mL) or FBS (10%). The media was removed, the cells washed with PBS, and the radioactive content of the media and cells determined by scintillation. Cholesterol efflux was determined by dividing the radioactive content of the media by the sum of the radioactivity in the cells and media.

RNA and protein analysis. RNA was isolated from cells with Trizol reagent, using a slight modification of the manufacturer's protocol to preserve miRNAs. Briefly, following homogenization in 2 mL of Trizol, 400 mL of chloroform and 200 mL if 2 mol/L sodium acetate pH 4.2 were added before the separation of the organic and aqueous phases. The latter was combined with 500 mL of acid phenol:chloroform:isoamylic acid, and centrifuged again. RNAs in the aqueous phase was then precipitated with 3 mL of ethanol overnight at 4° C. After centrifugation, the RNA pellet was resuspended in water and stored at −80° C. until use. Alternatively, RNAs were isolated using the mirVana miRNA Isolation Kit (Ambion). cDNAs were generated from 5 μg of DNase1-treated RNA using Superscript III and Random hexamers (Invitrogen). Real-time PCR was done with Sybr green reagent (Roche), using a LightCycler 480 real-time PCR detection system (Roche). Values were normalized to GAPDH and calculated using the comparative CT method. Protein levels were determined by Western blot, using 50 ug total liver protein extract, and a 1:1,000 dilution of anti-ABCA1 antibody (Novus Biologicals), following standard protocols.

Murine Studies. Male C57Bl/6J mice were obtained from Jackson Laboratories, and maintained in a 12 hour/12 hour light/dark cycle with unlimited access to chow and water. In examples 1-5, animals (n=6-8 per group) were infused with adenoviral vectors (2×10⁹ pfu) via tail vein injection. Blood was collected 5 days post-infusion, and total cholesterol and HDL-cholesterol were assayed enzymatically with the Cholesterol E and HDL-cholesterol E kits from Waco Chemicals. In example 6-10 animals (8-10 week old, n=6 per group, unless noted otherwise) were infused via tail vein injection with adenoviral vectors (2×10⁹ pfu) or antisense oligonucleotides (5 mpk, 2 consecutive days). Where indicated, mice were fed a 21% fat, 1% cholesterol, 0.5% cholate diet (Purina 5A8E). Tissues and bile were collected following overnight fasting. RCT experiments were performed as described 28. Briefly, mice were injected i.v. with oligonucleotides (5 mpk) twice for 2 weeks, and then injected i.p. with 1-1.5×10⁶ [³H]-cholesterol-loaded bone marrow-derived macrophages. Blood samples were collected at 6, 24 and 48 h after the injection of the cells. Liver, bile and the feces produced during the last 48 h were collected and flash-frozen until used. The amount of radiolabeled sterols was determined by scintillation. All studies involving mice were approved by the IACUC at Saint Louis University.

Luciferase Reporter Assays. Transient transfection of Hek293 cells was performed in triplicate in 24-well plates by the calcium phosphate method. Luciferase activity was measured 48 h after transfection using the Luciferase Assay System (Promega), and normalized to 3-galactosidase activity to correct for small changes in transfection efficiency.

Plasma Analysis, Circulating levels of ALT, AST, bilirubin and Natal bile acids were determined by Advanced Veterinary Laboratory (Saint Louis, Mo.).

Lipid Analysis, Liver (50 mg) was homogenized in 500 ml of PBS, and lipids were extracted from 100 ml of the liver homogenate in the presence of internal standards for each lipid class (Bligh et al., (1959) Can J Med Sci 37, 911-917). Similarly bile and plasma were extracted in the presence of internal standards for each lipid class. Id. Lipid species (e.g., phospholipids, triglycerides, cholesterol esters and ceramide) were quantified directly from lipid organic extracts using shotgun lipidomics that is based on class separation by MS/MS specific methods, the use of internal standards and response curves of natural compounds to the internal standards (Han and Gross, (2005) Mass Spectrom Rev 24, 367-412; Han (2002) Anal Biochem 302, 199-212; Han and Gross, (2001) Anal Biochem 295, 88-100; Ford, D. A., et al. (2008) J Neurochem 105, 1032-1047.

### Example 1

Expression of miR-33. In an attempt to identify miRNAs that might affect cholesterol homeostasis, in silico analysis were performed of human genes encoding nuclear receptors and transcription factors that were known to affect lipid homeostasis. The analysis identified sequences corresponding to miR-33 within intron 16 of human Srebp-2 (FIG. 2A). Importantly, these sequences are conserved across multiple animal species (FIG. 2B). To test the hypothesis that Srebp-2 and miR-33 are co-expressed and regulated by the same metabolic/sterol stimuli, mouse and human primary macrophages were incubated in media containing low or high sterols (see Methods), conditions that are known to regulate Srebp-2, SREBP-2 target genes and LXR target genes (Brown, and Goldstein, (1997) Cell; 89:331; Venkateswaran et al., (2000) Proc Natl Acad Sci USA; 97:12097). As expected, excess exogenous cholesterol resulted in increased expression of the LXR target genes Abca1 and Abcg1, with concomitant repression of SREBP-2 and its targets Hmgcr and Ldl-r, while the opposite pattern of gene expression was found in cells incubated in media lacking cholesterol (FIG. 2C, D, and data not shown). Importantly, miR-33 levels paralleled those of Srebp-2: they were repressed in cells incubated in high sterols and induced in cells incubated in media devoid of cholesterol (FIG. 2C, D).

### Example 2

miR-33 repression of LXR targeted Genes. Since SREBP-2 and LXR control antagonistic aspects of cellular sterol homeostasis, it was demonstrated that miR-33 was involved in repression of LXR target genes, such as ABCA1 and ABCG1 that are known to promote the efflux of cholesterol from cells. Analysis of the 3′-UTR regions of ABCA1 and ABCG1 identified sequences in both genes that are partially complementary to miR-33 sequences (FIGS. 3A, B, and C). In the case of human ABCA1, a proximal element at nucleotides 120-172 downstream of the stop codon was identified that contained three overlapping putative miR-33 responsive elements, and was termed Box 1. Also, a distal element at nucleotides 1,465-1,481 after the stop codon was identified and termed Box 2 (FIGS. 3A and C). For human ABCG1, a single putative miR-33 response element was identified at nucleotides 516-541 after the stop codon (FIGS. 33 and C). The same putative sequences were found in nucleotides 120-171 and 1,426-1,442, and 715-733 in the mouse ABCA1 and ABCG1 genes, respectively. Importantly, these sequences are evolutionarily conserved across animal species (FIG. 3A, B). To assess the functionality of these sequences, HEK293 cells were transfected with luciferase reporters containing the different miR-33 putative elements inserted after the stop codon. Co-transfection of a plasmid expressing miR-33 resulted in a 50-60% decrease in luciferase activity when the reporter plasmid contained the human/mouse Box 1 sequence from ABCA1 or the mouse ABCG1 sequence (FIG. 3D; lanes 5-6, 9-10). The specificity of this effect is supported by the finding that no repression was observed when the reporter gene contained either sequences corresponding to Box 2 of ABCA1 (FIG. 3D), or mutant Box 1 or mutant mouse ABCG1 sequences (FIG. 5). The miR-33 response element in the human ABCG1 gene is degenerate, as compared to the mouse and rat sequences, and does not confer miR-33 responsiveness (FIGS. 3B, C, and D: lanes 11-12). By comparison, a perfect miR-33 complementary sequence resulted in 95% repression of luciferase activity (FIG. 3D: lanes 3-4).

The ability of miR-33 to modulate the expression of endogenous ABCA1 and ABCG1 mRNAs was measured. Hep3B cells were transduced with an empty adenovirus or an adenovirus encoding miR-33 (FIG. 3E). In agreement with the data disclosed above, over-expression of miR-33 resulted in reduced expression of ABCA1, but not ABCG1 mRNA in these human hepatoma cells. Other lipid metabolism genes remained unaffected by miR-33 expression (FIG. 3E). Similar results were obtained in the human kidney-derived HEK293 cells transfected with miR-33 (data not shown). Collectively, these results identify human and murine ABCA1 and murine ABCG1 as bona-fide targets for miR-33.

### Example 3

miR-33 modulation of cellular sterol homeostasis. It was demonstrated that miR-33-dependent repression of ABCA1 and/or ABCG1 could affect cellular sterol homeostasis in vitro and in vivo. To demonstrate that miR-33 modulates the efflux of cellular cholesterol, HEK293 cells were transfected with scrambled or anti-miR-33 oligonucleotides (Dharmacon) and then incubated the with [3H]-cholesterol in the presence or absence of ligands for LXR and RXR to induce ABCA1 and ABCG1. The ability of the cells to efflux the radiolabeled sterol to BSA, ApoAI and FBS was analyzed 6 h later (FIG. 4). Following activation of LXR:RXR, silencing of miR-33 resulted in enhanced cholesterol efflux to ApoAI or FBS (FIG. 4A, compare lanes 5-6 to 7-8; and 9-10 to 11-12). Thus, mir-33 silencing increases the ability of cells to efflux cholesterol. These data offer a molecular explanation to previously reported studies showing that treatment with Statins, which induce Srebp-2, resulted in decreased expression of both ABCA1 and/or ABCG1 mRNAs, and attenuated cholesterol efflux in macrophages and endothelial cells (Wong, et al, (2007) Atherosclerosis; Apr. 25; Zeng et al., (2004) J Biol Chem.; 279:48801).

### Example 4

miR-33 modulation of physiological sterol homeostasis in vivo. To put these latter results in a physiological context, scrambled or anti-miR-33 oligonucleotides were injected into the tail veins of C57BL/6 mice (5 mg/Kg/day for 3 consecutive days), and plasma lipoprotein and cholesterol levels determined 12 days after the infusion. The data show that miR-33 silencing results in a 2-fold increase in hepatic ABCA1 mRNA and protein levels (FIG. 4B) and in a 10-15% increase in HDL-cholesterol, compared to mice treated with scrambled oligonucleotides (FIG. 4C). Collectively, these in vitro and in vivo studies demonstrate that miR-33 modulates intracellular cholesterol levels and ultimately plasma lipoprotein and cholesterol metabolism, presumably by silencing the expression of ABCA1 and/or ABCG1.

### Example 5

The Inventor discloses the following prolific example. Based on the preceding examples the inventor has demonstrated that by modulating levels of miR-33 in a subject, they were able to modulate plasma HDL cholesterol in vivo. More specifically, the inventor has demonstrated that the administration of an oligonucleotide encoding miR-33, via a viral vector, caused increased miR-33 levels, increased suppression of ABCA1, and decreased plasma HDL-cholesterol levels. One of ordinary skill in the art, using the compositions and methods described above, may administer, in a pharmaceutically acceptable carrier, an effective amount of antisense oligonucleotides complementary to miR-33, miR-33 preRNA, or miR-33 priRNA, such that functional levels of miR-33 will decrease, suppression of ABCA1 will decrease, and plasma HDL cholesterol levels will increase. The physiological effect will be to increase plasma levels of HDL-cholesterol as well as the HDL/LDL ratio and improved cardiovascular health.

### Example 6

Anti-miR-33 increases bile secretion in vivo. In an effort to understand the in vivo physiological importance of miR-33, the inventor injected mice with (5 mpk) of locked nucleic acid (LNA) scrambled sequence or LNA anti-miR-33 oligonucleotides 5-TGCAACTACAATGCA-3′ (SEQ ID NO: 8) in saline (0.9% NaCl) via tail vein injection, at 5 mg/Kg (mpk) body weight. Mice were then kept with unlimited access to water and regular chow for a week. The amount of bile collected from the gallbladders of fasted mice receiving anti-miR-33 oligonucleotides was almost double of that collected from control mice (FIG. 6A). Analysis of the pooled bile samples showed that the overall content of cholesterol and bile acids did not change between groups (FIG. 6B). However, the concentration of phosphatidylcholine was slightly elevated in the bile of mice receiving anti-miR-33 oligonucleotides (FIG. 6B). Next, the inventor tested the expression of several hepatocyte canalicular transporters that are known to mediate bile secretion. The inventor found that the mRNA levels of both Abcb11 and Atp8b1 were significantly increased in the livers of mice injected with anti-miR-33 oligonucleotides, compared to those receiving scrambled oligonucleotides (FIG. 6C). These results are specific, since mRNA levels of other canalicular transporters (Abcg5, Abcg8, Abcb4) remained unchanged (FIG. 6C). These results suggest that miR-33 controls bile secretion in mice by altering the expression of Abcb11 and Atp8b1.

### Example 7

ABCB11 and ATP8B1 have functional miR-33 responsive Sequences in the 3′UTR. Based on the results shown above, the inventor reasoned that both ABCB11 and ATP8B1 are direct targets of miR-33. Analysis of the 3′UTR of these genes revealed that sequences partially complementary to miR-33 are present in both ABCB11 (overlapping the stop codon for the human gene) and ATP8B1 (nucleotides 1877-1897 after the stop codon for the human gene) (FIGS. 7A and B). The importance of these sequences is evident in that these sequences are evolutionarily conserved for ATP8B1 (FIG. 7A). In the case of ABCB11 (FIG. 7B), this sequence is conserved among primates, while mice and rats lack this element but show a conserved sequence 732-751 nt after the stop codon; other rodents such as guinea pig have both the proximal and distal miR-33 sequences in the Abcb11 3′UTR (data not shown).

To test whether these sequences confer response to miR-33, the inventor cloned the 3′UTR of both human and mouse ATP8B1 and ABCB11, or the putative miR-33 responsive sequences, immediately downstream of a luciferase reporter. Co-transfection of these constructs into HEK293 cells in the presence or absence of a plasmid that overexpresses miR-33 confirmed that the 3′UTRs of these genes indeed respond to miR-33 (FIGS. 2C and 20). Hence, miR-33 overexpression resulted in ˜40% decrease in luciferase activity when the reporter is fused to the 3′UTR or the putative responsive elements of both human or mouse ATPB11 (FIG. 7C; lanes 5-8, and 11-14) or ABCB11 (FIG. 7D; lanes 1-4, and 7-10). As expected, mutations that prevent the binding of the seed sequence of the miRNA abolished the response to miR-33 (FIGS. 7C and D; lanes 9-10, and 15-16).

The inventor next sought to determine whether the endogenous mouse and human ABCB11 and ATP8B1 genes are regulated following miR-33 overexpression. To accomplish this, primary mouse hepatocytes obtained from 10 week-old male C57BL/6 mice were transduced with an empty adenovirus or an adenovirus encoding miR-33. Following 48 h incubation, total RNA were obtained, and the levels of different genes involved in lipid and bile metabolism examined. Data in FIG. 7E shows that the expression of Abcb11 and Atp8b1 is significantly reduced in cells following overexpression of miR-33 (FIG. 7E). These results are specific, since the expression of other canalicular transporters (Abcg5, Abcg8, Abcb4) and other bile-related genes (Fxr, Shp, Cyp7α, Cyp8b1) remained unchanged (FIG. 7E). As expected, similar results were obtained when using the human hepatocyte-derived cell line HepG2 (FIG. 7F). Taken together, data in FIGS. 6 and 7 identify human and mouse ATP8B1 and ABCB11 as functional direct targets of miR-33.

### Example 8

Silencing miR-33 rescues statin and diet-induced liver damage. Statins not only increase SREBP-2 expression but that they also increase miR-33 (Ho, et al., (2011) FASEB J. vol. 25 no. 5 1758-1766; Marquart, et al., (2010) Proc Natl Acad Sci USA 107, 12228-12232; Najafi-Shoushtari, et al., (2010) Science. Vol. 328 no. 5985 pp. 1566-1569; Rayner; et al., (2010) Science. Vol. 328 no, 5985 pp, 1570-1573) The inventor reasoned that statins may increase the risk of cholestasis by indirectly (via miR-33) repressing the expression of both ABCB11 and ATP8B1. To test this hypothesis, the inventor examined the combined effect of statins and cholestatic diet on liver function in mice. Specifically, the inventor gavaged chow-fed C57BL/6 animals (female, 10 week-old, n=6) simvastatin (0, 50, 150 or 300 mg/Kg/day) for two days prior to switching them to a 1% cholesterol, 0.5% cholate diet for an additional 7 days (the cholate diet). Simvastatin was administered daily during these latter 7 days (FIG. 9A). The inventor monitored body weight and food consumption during the length of the experiment. The inventor noted a dose-dependent lethality effect of simvastatin after mice were switched to the cholate diet (FIG. 9A). Hence, the health of mice on 300 mg/Kg (mpk) simvastatin precipitously declined by day 3 and all mice in this group had to be euthanized by day 5; on the other hand, all animals on 50 mpk simvastatin survived for the length of the experiment, while mice on 150 mpk simvastatin showed a 50% survival (FIG. 9A). This effect was paralleled by a dramatic dose-dependent increase in the relative weight of the livers: from 5.0±0.1% of body weight in control mice to 5.8±0.9% and 8.8±0.2% in mice receiving 50 and 150 mpk simvastatin, respectively (FIG. 9B). The livers of mice receiving 150 mpk simvastatin appeared not only enlarged, but also extremely steatotic (i.e. very pale, soft consistency) (FIG. 9C); in contrast, livers from mice receiving 50 mpk simvastatin looked similar to those from control mice (FIG. 9C). In all animals the gallbladders were dilated and filled with bile (FIG. 9C). ESI-MS analysis of the liver confirmed the accumulation of fatty acids, diglycerides and, remarkably, triglycerides in the livers of mice receiving 150 mpk simvastatin, compared to control animals (FIG. 9D). On the other hand, the amounts of cholesterol esters were significantly decreased in the 150 mpk group, even though unesterified cholesterol levels increased in the same livers (FIG. 9D). The overall ceramide content was decreased in these later animals (FIG. 9D); however, a closer examination of the individual species showed a profound remodeling of ceramides: those containing short fatty acids (16:0, 18:0, and 20:0) were increased in the livers of the 150 mpk group while those containing long chain fatty acids (23:0, 24:1, and 24:0) were significantly reduced in the same animals. In addition, bile acid levels were markedly elevated in the livers of mice receiving either 50 or 150 mpk simvastatin (FIG. 9D). Statin-induced hepatotoxicity was also apparent in the blood. Hence, the levels of ALT and AST transaminase enzymes, bilirubin, and bile acids increased in a dose-dependent manner (FIG. 9E), resulting in bright yellow plasma samples (FIG. 9E). These adverse effects of the drug were not the result of increased food intake, since mice gavaged with simvastatin eat significantly less food than control animals. Reduced food intake also reinforces the notion that simvastatin exerted toxic effects in these mice. Although the overall amount of be recovered from the gallbladder did not differ between groups (FIG. 9F), the inventor noted a dose-dependent decrease in phosphatidylcholine, a dramatic decrease in cholesterol in samples from the 150 mpk group, and no change in total bile acids (FIG. 9F). The inventor compared the expression of selected transcripts in the livers of mice gavaged saline or 50 mpk simnvastatin (FIG. 9G). The data shows that the levels of both Abcb11 and A1p8b1 were significantly reduced (˜40%) in the livers of mice receiving the drug; these changes were specific since the expression of other bile-related transporters (Abcb4, Abcg5, Abcg8) did not change between groups (FIG. 9G). These results prove conclusively that statins are pre-cholestatic in mice when combined with a cholate-rich diet. The levels of Srebp-2 and its two targets Hmgcr and Ldlr remained unchanged (FIG. 9G). The results from FIG. 9 support a mechanism in which statins induce miR-33, which in turn reduces the levels of both Abcb11 and Atp8b1, resulting in altered bile secretion from hepatocytes, which ultimately leads to cholestasis and liver malfunction.

### Example 9

Anti-miR-33 oligonucleotides provide benefits to statin induced BRIC or cholestasis. The inventor next tested whether silencing miR-33 could rescue mammals with statin induced ERIC or cholestasis. Mice (female, 10 week-old, n=10/group) received two consecutive doses of LNA scrambled or LNA anti-miR-33 oligonucleotide, (5′-TGCAACTACAATGCA-3′ (SEQ ID NO: 8), 5 mpk, saline, i.v.), and were then put on 150 mpk simnvastatin and fed the cholate diet (FIG. 10). Data show that, with one exception, all mice receiving anti-miR-33 oligonucleotides survived for at least a week. In contrast, mice injected with scrambled oligonucleotides exhibited <50% survival rate (FIG. 10A). Moreover, mice in which miR-33 was silenced had minimal loss of body weight, compared to animals receiving scrambled oligonucleotides (FIG. 10B). The livers from these mice appeared normal (i.e. non steatotic) (FIG. 10C), and the liver/body mass ratio was significantly lower than in control mice that succumbed to the diet and statin treatment (FIG. 10D). The inventor reasoned that the condition of the livers of those animals receiving scrambled oligonucleotides that were still alive at day 7 would have worsened had the treatment continued for a few more days. Plasma from mice receiving anti-miR-33 appeared clear (FIG. 10E). Rescue of the statin and diet-induced phenotype was also evident when the inventor analyzed the hepatic lipid contents: animals receiving anti-miR-33 oligonucleotides showed a marked decrease in free fatty acids, diglycerides, triglycerides and bile acids, compared to control animals (FIG. 10F). Finally, the inventor studied the mRNA levels of selected hepatic genes (FIG. 10G). Interestingly, the expression of all canalicular transporters, with the exception of Atp8b1, was severely decreased in mice that succumbed to the diet and statin; additionally, the expression of Abcg5 and Abcg8 varied tremendously among animals, independent of treatment (FIG. 10G). Comparing just those mice that survived at the end of the experiment, silencing miR-33 resulted in specific increased expression of Atp8b1 but not Abcb11 or any other canalicular transporter (FIG. 10G). Perhaps the expression of Abcb11 is already maximal in these livers, due to the activity of FXR. In general, the expression levels of the majority of bile-related genes in survivor mice in the scrambled group were closer to those in the antisense group (FIG. 10G), suggesting that the expression of these genes is critical for survival. Collectively, data in FIG. 10 show conclusively that miR-33 mediates diet- and statin-induced hepatotoxicity. Since SREBP-2/miR-33 are transcriptionally upregulated following treatment with statins (Ho et al., 2011; Marquart et al., 2010; Najafi-Shoushtari et al., 2010; Rayner et al., 2010), the inventor hypothesize that miR-33 might account for some of the pleiotropic, adverse effects of these drugs. Interestingly, several recent reports show that some patients following a prescription of statins rapidly develop cholestasis (Batey and Harvey, (2002) Med J Aust 176, 561; de Castro et al., (2006) Hepatol 29, 21-24; Merli et al., (2010) Clin Drug Investig 30, 205-209; Rahier et al., (2008) Acta Gastroenterol Belg 71, 318-320; Ridruejo and Mando, (2002) J Hepatol 37, 165-166; Torres at al., (2002) Med Clin (Bare) 118, 717). While the molecular events that occur in the livers of these patients are unknown, this clinical setting is consistent with our model (FIG. 7B) in which statin-induced miR-33 downregulates the expression of both ABCB11 and ATP8B1 (and perhaps, indirectly, also ABCG5/ABCG8), thus decreasing bile secretion and eventually leading to cholestasis. In general, most physicians are cautious to prescribe statins to patients with underlying liver diseases, although recent reports suggest that statins might provide protection from liver damage to patients with primary biliary cirrhosis (Abu Rajab and Kaplan, (2010) Dig Dis Sci 55, 2086-2088; Stojakovic et al., (2010) Atherosclerosis 209, 178-183) and to rodents following bile duct ligation (Awad and Kamel, (2010) J Biochem Mol Toxicol 24, 89-94; Demirbilek et al., (2007) Pediatr Surg Int 23, 155-162; Dold at al., (2009) Br J Pharmacol 156, 466-474). Other, however, have reported that statins have no beneficial effects in patients with primary biliary cirrhosis (Stanca et al., (2008) Dig Dis Sci 53, 1988-1993; Stojakovic et al., (2007) Hepatology 46, 776-784). In any case, it is safe to assume that, at the relative low doses normally prescribed, the benefits of statins outweigh the unlikely side effects in patients with pre-existing liver conditions (Bader, (2010) Am J Gastroenterol 105, 978-980). Nevertheless, the inventor has shown that a dose-dependent effect of simvastatin on diet-induced hepatotoxicity and cholestasis, which can be rescued by silencing miR-33 with oligonucleotides. Based on the above results, the inventor discloses a method of treatment using anti-miR-33 oligonucleotides to manage patients who develop BRIC as a consequence of partial loss of function of ABCB11 or ATP8B1, or patients in which cholestasis appears following the prescription of statins.

### Example 10

ANTI-miR-33 Treatment Results In Increased Reverse Cholesterol Transport. Biliary secretion is an essential component of the reverse cholesterol transport (RCT) pathway (Nijstad at al., (2011) Gastroenterology 140, 1043-1051) by which extrahepatic cholesterol is shuttled to the liver, secreted into bile and excreted through feces (Khera and Rader, (2010) Curr Atheroscler Rep 12, 73-81; Rader et al., (2009) J Lipid Res 50 Suppl, S189-194; Wang et al., (2007) J Clin Invest 117, 2216-2224; Wang and Rader, (2007) J Biol Chem 280, 8742-8747). An additional, liver-independent ROT pathway has been proposed that removes circulating cholesterol through the intestine directly into feces (van der Veen et al., (2009) J Biol Chem 284, 19211-19219). The inventor reasoned that the changes in be secretion observed following manipulation of miR-33 levels will result in altered RCT. Also, a previously described target of miR-33, ABCA1 (Gerin at al., (2010) J Biol Chem 285, 33652-33661; Horie et al., (2010) Proc Natl Acad Sci USA 107, 17321-17326; Marquart et al., (2010) Proc Natl Acad Sci USA 107, 12228-12232; Najafi-Shoushtari et al., (2010) Science. Vol. 328 no. 5985 pp. 1566-1569; Rayner at al., (2010) Science, Vol. 328 no. 5985 pp. 1570-1573), has been shown to play an essential role for ROT (Wang et al., (2007) J Clin Invest 117, 2216-2224). To demonstrate, the inventor injected male C57BL/6 mice (n=6/group) with macrophage foam cells that were radiolabeled with tritiated cholesterol, and followed the destiny of the labeled sterols for 48 h (see Experimental Procedures for details). The inventor did not observe changes in body, liver of feces mass. Data show that the amount of labeled cholesterol in circulation increased in mice receiving LNA anti-miR-33 oligonucleotide, (5 mpk) 5′-TGCAACTACAATGCA-3′ (SEQ ID NO; 8), compared to control animals (FIG. 8A). However, the amount of labeled sterols found in the liver did not statistically differ between mice (FIG. 8B). Analysis of the bile recovered from the gallbladder confirmed that bile secretion is increased after suppression of miR-33 expression. Moreover, the amount of labeled sterols recovered from the gallbladder was increased in these latter mice (FIG. 8C); importantly, even when corrected for volume, the data show that the contents of labeled biliary sterols (i.e. dpm/μL of bile) are increased 2-fold as compared to mice receiving anti-miR-33 oligonucleotides. Finally, the recovery of labeled sterols in the feces increased ˜2-fold in these same mice, compared to control animals (FIG. 8D). Taken together, these data demonstrate that miR-33 modulates ROT, likely through the combined regulation of ABCA1, ABCB11 and ATP8B1.

All publications and patents cited in this specification are hereby incorporated by reference in their entirety. The discussion of the references herein is intended merely to summarize the assertions made by the authors and no admission is made that any reference constitutes prior art. Applicants reserve the right to challenge the accuracy and pertinence of the cited references.

What is claimed is:

1. A method of treating a statin induced secondary effect comprising, administering an effective amount of an antisense compound complementary to miR-33, to a subject.

2. The method of claim 1, whereby the statin induced secondary effect is selected from the group consisting of raised liver enzymes, rhabdomyolysis, cognitive loss, neuropathy, pancreatic, hepatic dysfunction, and sexual dysfunction.

3. The method of claim 1, whereby the statin induced secondary effect is cholestasis.

4. The method of claim 1, whereby the statin induced secondary effect is Benign Recurrent Intrahepatic Cholestasis.

5. The method of claim 1, whereby the statin is selected from the group consisting of atorvastatin, cerivastatin, fluvastatin, lovastatin, mevastatin, pitavastatin, pravastatin, rosuvastatin, and simvastatin.

6. The method of claim 1, whereby the antisense compound consists of 8 or more contiguous nucleotide-bases complementary to SEQ ID NO: 4.

7. The method of claim 1, whereby the antisense compound consists of 10 or more contiguous nucleotide-bases set forth in SEQ ID NO: 5.

8. The method of claim 1, whereby the antisense compound consists of the sequence set forth in SEQ ID NO: 5.

9. The method of claim 1, whereby the antisense compound consists of the sequence set forth SEQ ID NO:8.

10. The method of claim 1, whereby the subject is a human patient in need.

11. A method of treating Benign Recurrent Intrahepatic Cholestasis not associated with statins comprising, administering an effective amount of an antisense compound complementary to miR-33, to a subject.

12. The method of claim 11, whereby the antisense compound consists of 8 or more contiguous nucleotide-bases complementary to SEQ ID NO: 4.

13. The method of claim 11, whereby the antisense compound consists of 10 or more contiguous nucleotide-bases set forth in SEQ ID NO:5.

14. The method of claim 11, whereby the antisense compound consists of the sequence set forth SEQ ID NO:8.

15. The method of claim 11, whereby the subject is a human patient in need.

16. A method of improving cardiovascular health in a subject by increasing reverse cholesterol transport (RCT) in a subject comprising, administering an effective amount of an antisense compound complementary to miR-33.

17. The method of claim 16, whereby the antisense compound consists of 8 or more contiguous nucleotide-bases complementary to SEQ ID NO: 4.

18. The method of claim 16, whereby the antisense compound consists of 10 or more contiguous nucleotide-bases set forth in SEQ ID NO:5.

19. The method of claim 16, whereby the antisense compound consists of the sequence set forth SEQ ID NO:8.

20. The method of claim 16, whereby the subject is a human patient in need.


SEQUENCE LISTING (8 sequences)

SEQ ID NO: 1 - RNA, 54 nt, Homo sapiens
uguacugauacuauucaaugcaaugcaauucaaugcaaugaaaacaaaauucca

SEQ ID NO: 2 - RNA, 18 nt, Homo sapiens
uuaauugcaacaaugcag

SEQ ID NO: 3 - RNA, 27 nt, Homo sapiens
ugcaagccaaaagucgaucaaucgcau

SEQ ID NO: 4 - RNA, 21 nt, Homo sapiens
gugcauuguaguugcauugca

SEQ ID NO: 5 - DNA, 21 bp, Homo sapiens
tgcaatgcaactacaatgcac

SEQ ID NO: 6 - RNA, 69 nt, Homo sapiens
cuguggugcauuguaguugcauugcauguucuggugguacccaugcaauguuuccacagugcaucacag

SEQ ID NO: 7 - DNA, 69 bp, Homo sapiens
ctgtgatgcactgtggaaacattgcatgggtaccaccagaacatgcaatgcaactacaatgcaccacag

SEQ ID NO: 8 - DNA, 15 bp, Homo sapiens
tgcaactacaatgca